(19) United States
(12) Patent Application Publication
(10) Pub. No.: US 2020/0385614 A1
(43) Pub. Date: Dec. 10, 2020

(54) DUAL ELECTROMAGNETIC RADIATION LIGHT SWITCHABLE ADHESIVE AND APPARATUSES, SYSTEMS, AND METHODS THEREFORE

(71) Applicant: KCI LICENSING, INC., San Antonio, TX (US)

(72) Inventors: Christopher Brian LOCKE, Bournemouth (GB); Timothy Mark ROBINSON, Blandford Forum (GB)

(21) Appl. No.: 16/567,178

(22) Filed: Sep. 11, 2019

Related U.S. Application Data
(60) Provisional application No. 62/858,089, filed on Jun. 6, 2019.

(51) Int. Cl. C09J 11/06; C09J 9/00; C09J 133/08; C09J 175/04; A61L 15/58

(52) U.S. Cl. CPC C09J 11/06; C09J 9/00; C09J 133/08; C08K 5/5397; A61L 15/585; C09J 2205/31; C09J 175/04

(57) ABSTRACT

This disclosure describes devices, systems, and methods related to light switchable adhesives, the manufacture thereof, and the use thereof. An exemplary light switchable adhesive includes one or more polymers, first photo initiators configured to cause the one or more polymers to cross-link responsive to receiving first light, and second photo initiators configured to cause the one or more polymers to cross-link responsive to receiving second light, the second photo initiators different from the first photo initiators.

### CROSS-REFERENCE TO RELATED APPLICATIONS

This application claims priority to and the benefit of U.S. Provisional Application No. 62/858,089, filed Jun. 6, 2019, which is incorporated into the present application by reference.

### TECHNICAL FIELD

Aspects of the present disclosure relate generally to a light switchable adhesive, such as a light switchable adhesive for use with medical devices, and more specifically, but not by way of limitation, to an apparatus including the light switchable adhesive and an apparatus, system, and method for forming the light switchable adhesive.

### BACKGROUND

Light switchable (switched or light switched) adhesives are pressure sensitive adhesives that are “switchable” from a tacky state to a non-tacky or low-tack state in which the switched adhesive has a reduced peel strength relative to the peel strength of the adhesive before switching. Conventional light switchable adhesives have two states or phases (e.g., off and on). In the first state, the light switchable adhesive is tacky and sticks to itself and other objects. Once the conventional light switchable adhesive is switched or activated, the conventional light switchable adhesive cross-links and becomes brittle and hard, which reduces the tackiness and peel strength. The cross-linking is an irreversible chemical process and thus, a conventional light switchable adhesive can only be switched once, from a high tact/peel strength state to a lower tact/peel strength state. Light switchable adhesives have been incorporated into many products, including some medical devices.

When applying medical devices, such as a dressing, the dressing is often repositioned during an initial application of the dressing or the dressing comes into inadvertent contact with an unintended object during application. With conventional adhesives and light switchable adhesives, the dressing and adhesive are applied to the patient in a high tact/peel strength state. Thus, a patient may have to go back to the emergency room and undergo anesthesia to have the dressing repositioned/removed or a light switchable adhesive of the dressing is activated to facilitate easier removal and the dressing is wasted. Similarly, when a conventional acrylic adhesive or conventional light switchable adhesive comes into contact with itself or another component (i.e., not the intended bond site) during application, the components often become inseparable and cannot be separated without damaging the components and/or the adhesive, and thus the components are wasted. As an illustrative example, drape layers can be susceptible to such problems. For example, the drape layer is often thin and has a tendency to bunch up, causing the conventional light switchable adhesive to stick to itself and the drape.

While silicone adhesives may be reworked and repositioned during application, including worked into creases of the tissue or to accommodate complex shapes, and may be recoverable from unintended contact, silicone adhesives do not offer sufficient bond/peel strength and wearability time as compared to conventional acrylic adhesives or conventional light switchable adhesives. Additionally, in some medical applications components (e.g., single use components) may be assembled incorrectly. In such cases, the components cannot be disassembled and reconnected because the conventional acrylic adhesive or the conventional light switchable adhesive is not reusable.

Therefore, conventional light switchable adhesive applications are not repositionable and are harder and less forgiving to apply as compared to silicone adhesives. As a result conventional light switchable adhesives and medical devices that incorporate such conventional light switchable adhesives can be painful or impossible to reposition and/or reapply.

### SUMMARY

This disclosure describes light switchable adhesives, devices including light switchable adhesives, and systems and methods related to forming and/or using light switchable adhesives. The light switchable adhesives described herein include more than two phases, such as three or more phases, also referred to herein as states, and are activated by two different type of light (electromagnetic radiation). For example, the light switchable adhesives described herein include multiple types of photo initiators which transition the light switchable adhesive between phases multiple times. To illustrate, the light switchable adhesives described herein include (i.e., are transitionable between) a first phase with a low peel strength state and tact, a second phase with a relatively higher peel strength, and a third phase with a relatively lower peel strength. The multiple types of photo initiators can be used (e.g., by activation with light) to transition the light switchable adhesive between the first, second, and third phases. Accordingly, the light switchable adhesives described herein can be repositioned and are resistant to inadvertent contact.

To illustrate, the first phase is a partially cured or uncured state and has a low peel strength and tact. In the first phase, the light switchable adhesive can be repositioned because it has a low peel strength and tact as compared to conventional light switchable adhesives and acrylic adhesives. As an analogy, the light switchable adhesives described herein behave more like silicone adhesives than conventional light switchable adhesives and acrylic adhesives when in the first phase. However, the light switchable adhesives described herein can be cured or increased in a curing amount or degree by applying first light to increase a peel strength and tact of the light switchable adhesive. Thus, the light switchable adhesive, once placed in or worked into a desired position, can be set or cured to a second state (an operational state) where the light switchable adhesive has a peel strength and tact similar to conventional light switchable adhesives and acrylic adhesives.

Similar to conventional light switchable adhesives, the light switchable adhesives described herein can be “deactivated” to achieve a lower peel strength responsive to receiving second light (e.g., light having a different wavelength than the first light). To illustrate, the light switchable adhesives described herein receive second light and transition from the second phase to a third phase where the light switchable adhesive can be removed. Accordingly, the light switchable adhesives described herein offer stronger peel strengths and longer wear times than silicone adhesives, while offering silicone adhesive like benefits of repositioning with lower tact. The light switchable adhesives describe herein are easier to use than acrylic adhesives and conventional light switchable adhesives because they include an extra phase with a lower tact and peel strength to facilitate easier application and repositioning.

In some implementations, a light switchable adhesive is included in a compound film. An exemplary compound film may include two layers of polymer materials, where the layers are separable or removable. A removable layer can block or filter wavelengths of light that would otherwise activate second photo initiators of the light switchable adhesive and can pass or transmit light that is capable of activating first photo initiators of the light switchable adhesive. The compound film may be included in medical device to provide for pain and trauma free removal of wound dressing and/or single use connections between components.

Thus, the light switchable adhesives of the present disclose are configured to have an additional phase or state, as compared to conventional two phase light switchable adhesives. Accordingly, such light switchable adhesives can be repositioned and are more resilient to inadvertent contact or improper assembly than conventional two phase light switchable adhesives. Therefore, the light switchable adhesives described herein are suitable for use in medical devices, such as bandages, drapes, dressings, and wound closures. The light switchable adhesives enable medical devices to be repositioned and removed easily, thereby avoiding or limiting maceration and tissue damage at a tissue site and patient discomfort. Accordingly, the light switchable adhesives may enable improved wound care and therapy and increased wear times of medical devices, thereby advancing patient comfort and confidence in the treatment.

Some embodiments of the present compositions (e.g., a light switchable adhesive) comprise: one or more polymers; first photo initiators configured to cause the one or more polymers to cross-link responsive to receiving first light; and second photo initiators configured to cause the one or more polymers to cross-link responsive to receiving second light, the second photo initiators different from the first photo initiators.

In some of the foregoing embodiments of the present compositions, the light switchable adhesive has at least three phases, each phase corresponding to a particular peel strength, and wherein the light switchable adhesive is configured to transition between a first two phases of the three phases based on activation of the first photo initiators and to transition between a second two phases of the three phases based on activation of the second photo initiators. In some implementations, the light switchable adhesive has a second peel strength in the second phase that is greater than a first peel strength of the light switchable adhesive in the first phase, and wherein a third peel strength of the light switchable adhesive in the third phase is less than the second peel strength.

In some of the foregoing embodiments of the present compositions, the one or more polymers include an Acrylate polymer, and wherein the light switchable adhesive comprises a polymer composition that includes the one or more polymers, the first photo initiators, and the second photo initiators. In some implementations, the one or more polymers include urethane acrylate, methyl acrylate, silicone acrylate, polyether, polyurethane, or a combination thereof.

In some of the foregoing embodiments of the present compositions, the first photo initiators have a peak absorbance between 750 nanometers (nm) to 860 nm. In some implementations, the second photo initiators have a peak absorbance between 200 nanometers (nm) to 400 nm. In some of the foregoing embodiments of the present compositions, the first photo initiators comprise H-Nu-IR 780, H-Nu-IR 815, or both. In some implementations, the second photo initiators comprise Irgacure 819.

In some of the foregoing embodiments of the present compositions, the light switchable adhesive has a peel strength of less than 7 N/25 mm on stainless steel at an angle of 180 degrees in a first phase. In some implementations, the light switchable adhesive has a peel strength of greater 8 N/25 mm on stainless steel at an angle of 180 degrees in a second phase. Additionally, or alternatively, the light switchable adhesive has a peel strength of less than 7 N/25 mm on stainless steel at an angle of 180 degrees in a third phase.

In some of the foregoing embodiments of the present compositions, the light switchable adhesive has an areal weight of 100 to 250 grams per square meter (gsm). In some implementations, the light switchable adhesive has a moisture vapor transfer rate (MVTR) of greater than 250 g/m². Additionally, or alternatively, the light switchable adhesive has a viscosity which produces 7 mm to 11 mm of cone penetration according to ISO 2137 (e.g., 70 mm/10 to 110 mm/10).

In some of the foregoing embodiments of the present compositions, the light switchable adhesive has a second level of cross-linking in the second phase that is greater than a first level of cross-linking in the first phase, and wherein the light switchable adhesive has a third level of cross-linking in the third phase that is greater than the second level of cross-linking.

Some embodiments of the present apparatuses (e.g., a compound film) comprise: a first layer of a first polymer composition; a second layer of a second polymer composition, the second layer removeably coupled to the first layer; and a light switchable adhesive of the foregoing embodiments coupled to the second polymer layer. In some of the foregoing embodiments of the present apparatuses: the first layer is in direct contact with the second layer; and the light switchable adhesive is in direct contact with the second layer. In some implementations, the first layer is opaque and the second layer is optically transparent.

In some of the foregoing embodiments of the present apparatuses: the first layer is configured to block or filter ultraviolet (UV) light, visible light, or both; and the second layer is configured to pass UV light, visible light, or both. In some implementations, the second layer is configured to diffuse UV light, visible light, or both. In some of the foregoing embodiments of the present apparatuses: the second layer is configured to pass visible light, infrared light, or both; and the first layer is configured to block or filter visible light.

In some of the foregoing embodiments of the present apparatuses, the apparatuses further comprise a cover film removeably coupled to the light switchable adhesive. In some implementations, the first layer is included in a drape, a bandage, a wound closure device, a therapy system adhesive, or a combination thereof.

In some of the foregoing embodiments of the present apparatuses, the light switchable adhesive, the first layer, the second layer, or a combination thereof, define a plurality of perforations. In some implementations, the light switchable adhesive is arranged in a pattern.

Some embodiments of the present systems comprise: a medical device including the light switchable adhesive of any of the foregoing embodiments. In some of the foregoing embodiments of the present systems, the systems further comprise a therapy device coupled to the medical device and configured to provide therapy via the medical device. In some implementations, the systems further comprise a light source configured to emit light to the light switchable adhesive to transition the light switchable adhesive from a first phase to a second phase.

In some of the foregoing embodiments of the present systems, the medical device comprises a wound dressing, a bandage, or a wound closure device. In some implementations, the light switchable adhesive is coupled to a compound film, and the compound film corresponds to a protective film and a drape layer of the wound dressing. In other implementations, the medical device comprises a connector of the system, and the light switchable adhesive is coupled an interface of the connector and is configured to form a connection point between two components of the system.

Some embodiments of the present methods of manufacturing light switchable adhesive comprise: providing first photo initiators to one or more polymers, the first photo initiators configured to increase a cross-linking of the one or more polymers responsive to receiving first light; providing second photo initiators to the one or more polymers, the second photo initiators different from the first photo initiators and configured to increase the cross-linking of the one or more polymers responsive to receiving second light; and blending the one or more polymers, the first photo initiators, and the second photo initiators to form a polymer composition.

In some of the foregoing embodiments of the present methods, the methods further comprise providing one or more co-initiators configured to assist the first photo initiators, the first photo initiators, or both, in curing the one or more polymers. In some implementations, the one or more the co-initiators include Borate V, Irgacure 184, or both.

In some of the foregoing embodiments of the present methods, the methods further comprise providing one or more solvents configured to increase a solubility of the first photo initiators, the first photo initiators, or both, in the one or more polymers. In some implementations, the one or more solvents include N,N-Dimethylacrylamide (DMAA), ketones, or both. In some of the foregoing embodiments of the present methods, the methods further comprise partially curing the polymer composition by applying heat, light, or both.

In some of the foregoing embodiments of the present methods, the polymer composition comprises a light switchable adhesive, and the methods further comprise applying the light switchable adhesive to a film. In some implementations, applying the light switchable adhesive to the film includes applying a coating of the light switchable adhesive by a roller, a slot die, or a spray nozzle. In a particular implementation, applying the light switchable adhesive to the film includes applying the light switchable adhesive in a pattern. Additionally, or alternatively, the methods may further comprise coupling a cover film to the light switchable adhesive. In some of the foregoing embodiments of the present methods, the methods further comprise forming perforations in the light switchable adhesive, the cover film, or a combination thereof

Some embodiments of the present methods of using light switchable adhesive comprise: attaching a component to a tissue site via a light switchable adhesive to form a bond between the component and the tissue site; applying first light to the light switchable adhesive to increase a bond strength of the bond between the component and the tissue site; applying second light to the light switchable adhesive to decrease the bond strength of the bond between the component and the tissue site; and removing the component from the tissue site.

In some of the foregoing embodiments of the present methods, the methods further comprise, prior to attaching the component, removing a cover film from the light switchable adhesive. In some implementations, the methods further comprise, after applying the first light, removing a protective film from the component, wherein removing the protective film enables application of the second light to the light switchable adhesive.

Some embodiments of the present methods of using light switchable adhesive comprise: receiving, at a light switchable adhesive of a component, first light; responsive to receiving the first light, transitioning, by the light switchable adhesive, from a first phase to a second phase; receiving, at the light switchable adhesive, second light, the second light different from the first light; and responsive to receiving the second light, transitioning, by the light switchable adhesive, from the second phase to a third phase.

In some of the foregoing embodiments of the present methods, the methods further comprise, after receiving the second light, decreasing, by the light switchable adhesive, a bond strength between the component and a tissue site. In some implementations, the methods further comprise, after receiving the second light, debonding, by the light switchable adhesive, the component from a tissue site, wherein the third phase has a lower peel strength than the second phase.

In some of the foregoing embodiments of the present methods, the methods further comprise, responsive receiving the first light or the second light, changing color by the light switchable adhesive. In some implementations, the methods further comprise, prior to receiving the first light, bonding, by the light switchable adhesive, the component to a tissue site. Additionally, or alternatively, the methods further comprise, after receiving the first light, increasing, by the light switchable adhesive, a bond strength between the component and the tissue site.

Some embodiments of the present methods of using light switchable adhesive comprise: emitting first light by a light device, the first light configured to cause a light switchable adhesive to transition from a first phase to a second phase; and emitting second light by the light device, the second light different from the first light and configured to cause the light switchable adhesive to transition from the second phase to a third phase.

In some of the foregoing embodiments of the present methods, the methods further comprise: emitting reference light; determining a distance to a surface based on the reference light; and outputting an indication of the determined distance, wherein the first light comprises one of UV light, visible light, or infrared light, and wherein the second light comprises another of UV light, visible light, or infrared light. In a particular implementation, the first light comprises IR light, and wherein the second light comprises UV light.

In some of the foregoing embodiments of the present methods, the first light has a wavelength between 650 nanometers (nm) and 850 nm. Additionally, or alternatively, the second light has as a wavelength between 200 nanometers (nm) and 450 nm.

Some embodiments of the present kits (e.g., a kit for a light switchable adhesive) comprise: a three phase light switchable adhesive configured to transition between a first phase and a second phase responsive to receiving first light and to transition between the second phase and a third phase responsive to receive second light, the second light different from the first light.

In some of the foregoing embodiments of the present kits, the first light corresponds to infrared light and the second light corresponds to ultraviolet light, and the kits further comprise a dual light device configured to emit infrared light and emit ultraviolet light. Additionally, or alternatively, the kits further comprise a package that includes the three phase light switchable adhesive.

Some embodiments of the present kits (e.g., a kit for a light switchable adhesive) comprise: a dual light device configured to emit infrared light and ultraviolet light, the dual light device comprising: an infrared light source configured to emit the infrared light; and an ultraviolet light source configured to the ultraviolet light.

In some of the foregoing embodiments of the present kits, the dual light device further comprises a distance indicator device. In some implementations, the distance indicator device comprises: two light sources configured to emit reference light; a controller configured to determine a distance between the light sources and a surface based on the reference light; and an indicator configured to provide an indication of the determined distance.

In some of the foregoing embodiments of the present kits, the kits further comprise a three phase light switchable adhesive. In some implementations, the kits further comprise a package that includes the dual light device.

As used herein, the term “switchable” will be used to refer to adhesives which can be changed at least from one phase (e.g., a high tack and/or peel strength phase, also referred to as a state) to another phase (e.g., a low tack and/or peel strength phase, such as a non-tacky phase). Recognizing that the expression “low tack and/or peel strength” is a relative term, it will be defined here as meaning a condition of a minimum reduction in tackiness which the adhesive reaches after switching from the high tack and/or peel strength phase. The reduction in tack or peel force may be as great as 99% or as little as 30%. Typically, the reduction in tack or peel force is between 70% and 90%.

As used herein, the term “peel strength” will be used to refer to a strength of adhesives measured by a 180 degree angle peel test on stainless steel at room temperature. Recognizing that a bond strength of adhesive depends on the medium to which it adheres and that tissue composition can vary greatly, the measured peel strength is indicative of the adhesive's bond strength with tissue.

As used herein, various terminology is for the purpose of describing particular implementations only and is not intended to be limiting of implementations. For example, as used herein, an ordinal term (e.g., “first,” “second,” “third,” etc.) used to modify an element, such as a structure, a component, an operation, etc., does not by itself indicate any priority or order of the element with respect to another element, but rather merely distinguishes the element from another element having a same name (but for use of the ordinal term). The term “coupled” is defined as connected, although not necessarily directly, and not necessarily mechanically. Additionally, two items that are “coupled” may be unitary with each other. To illustrate, components may be coupled by virtue of physical proximity, being integral to a single structure, or being formed from the same piece of material. Coupling may also include mechanical, thermal, electrical, communicational (e.g., wired or wireless), or chemical coupling (such as a chemical bond) in some contexts.

The terms “a” and “an” are defined as one or more unless this disclosure explicitly requires otherwise. The term “substantially” is defined as largely but not necessarily wholly what is specified (and includes what is specified; e.g., substantially 90 degrees includes 90 degrees and substantially parallel includes parallel), as understood by a person of ordinary skill in the art. As used herein, the term “approximately” may be substituted with “within 10 percent of” what is specified. Additionally, the term “substantially” may be substituted with “within [a percentage] of” what is specified, where the percentage includes 0.1, 1, or 5 percent; or may be understood to mean with a design, manufacture, or measurement tolerance. The phrase “and/or” means and or. To illustrate, A, B, and/or C includes: A alone, B alone, C alone, a combination of A and B, a combination of A and C, a combination of B and C, or a combination of A, B, and C. In other words, “and/or” operates as an inclusive or.

The terms “comprise” (and any form of comprise, such as “comprises” and “comprising”), “have” (and any form of have, such as “has” and “having”), and “include” (and any form of include, such as “includes” and “including”). As a result, an apparatus that “comprises,” “has,” or “includes” one or more elements possesses those one or more elements, but is not limited to possessing only those one or more elements. Likewise, a method that “comprises,” “has,” or “includes” one or more steps possesses those one or more steps, but is not limited to possessing only those one or more steps.

Any aspect of any of the systems, methods, and article of manufacture can consist of or consist essentially of—rather than comprise/have/include—any of the described steps, elements, and/or features. Thus, in any of the claims, the term “consisting of” or “consisting essentially of” can be substituted for any of the open-ended linking verbs recited above, in order to change the scope of a given claim from what it would otherwise be using the open-ended linking verb. Additionally, it will be understood that the term “wherein” may be used interchangeably with “where.”

Further, a device or system that is configured in a certain way is configured in at least that way, but it can also be configured in other ways than those specifically described. The feature or features of one embodiment may be applied to other embodiments, even though not described or illustrated, unless expressly prohibited by this disclosure or the nature of the embodiments.

Some details associated with the aspects of the present disclosure are described above, and others are described below. Other implementations, advantages, and features of the present disclosure will become apparent after review of the entire application, including the following sections: Brief Description of the Drawings, Detailed Description, and the Claims.

### BRIEF DESCRIPTION OF THE DRAWINGS

A further understanding of the nature and advantages of the present disclosure may be realized by reference to the following drawings. The following drawings illustrate by way of example and not limitation. For the sake of brevity and clarity, every feature of a given structure is not always labeled in every figure in which that structure appears. Identical reference numbers do not necessarily indicate an identical structure. Rather, the same reference number may be used to indicate a similar feature or a feature with similar functionality, as may non-identical reference numbers.

FIG. 1A is a block diagram of an example of a system for activating a light switchable adhesive;

FIG. 1B is a side view of an example of a compound film including a light switchable adhesive;

FIG. 2A is diagram illustrating representative chemical reactions of a light switchable adhesive;

FIGS. 2B-2D are each a representative chemical view of phases of an example of a light switchable adhesive;

FIG. 3 is diagram illustrating peel strength of a light switchable adhesive and a degree of cross-linking of the light switchable adhesive;

FIGS. 4A-4D are each a side view of an example of attaching and removing a compound film including light switchable adhesive from tissue;

FIGS. 5A-5C are each a diagram illustrating a chemical formula of an example of a photo initiator of a light switchable adhesive;

FIG. 6A is a diagram of an example of a therapy system including a light switchable adhesive;

FIG. 6B is a diagram of an example of a drape of the therapy system of FIG. 6A including a light switchable adhesive;

FIG. 7 is a block diagram of a manufacturing system for manufacturing a light switchable adhesive and coating objects with the light switchable adhesive;

FIG. 8 is a block diagram of an example of a kit for light switchable adhesives;

FIG. 9 is a flowchart illustrating an example of a method of manufacturing light switchable adhesive;

FIG. 10 is a flowchart illustrating an example of a method of using light switchable adhesive;

FIG. 11 is a flowchart illustrating an example of another method of using light switchable adhesive; and

FIG. 12 is a flowchart illustrating an example of a method of using a light device to activate a light switchable adhesive.

### DETAILED DESCRIPTION

As used herein, the terms “tissue site” and “target tissue” as used herein can broadly refer to a wound (e.g., open or closed), a tissue disorder, and/or the like located on or within tissue, such as, for example, bone tissue, adipose tissue, muscle tissue, neural tissue, dermal tissue, vascular tissue, connective tissue, cartilage, tendons, ligaments, and/or the like. The terms “tissue site” and “target tissue” as used herein can also refer to a surrounding tissue area(s) and/or areas of tissue that are not necessarily wounded or exhibit a disorder, but include tissue that would benefit from tissue generation and/or tissue that may be harvested and transplanted to another tissue location. The terms “tissue site” and “target tissue” may also include incisions, such as a surgical incision. In some implementations, “target tissue” may correspond or refer to a wound, and “tissue site” may correspond or refer to a tissue area(s) surrounding and including the target tissue. Additionally, the term “wound” as used herein can refer to a chronic, subacute, acute, traumatic, and/or dehisced incision, laceration, puncture, avulsion, and/or the like, a partial-thickness and/or full thickness burn, an ulcer (e.g., diabetic, pressure, venous, and/or the like), flap, and/or graft. A wound may include chronic, acute, traumatic, subacute, and dehisced wounds, partial-thickness burns, ulcers (such as diabetic, pressure, or venous insufficiency ulcers), flaps, grafts, and fistulas, for example.

The term “positive-pressure” (or “hyperbaric”) as used herein generally refers to a pressure greater than a local ambient pressure, such as the ambient pressure in a local environment external to a sealed therapeutic environment (e.g., an internal volume). In most cases, this positive-pressure will be greater than the atmospheric pressure at which the patient is located. Alternatively, the positive-pressure may be greater than a hydrostatic pressure associated with tissue at the tissue site. Unless otherwise indicated, values of pressure stated herein are gauge pressures. References to increases in positive-pressure typically refer to an increase in absolute pressure, and decreases in positive-pressure typically refer to a decrease in absolute pressure. Additionally, the process of increasing pressure may be described illustratively herein as “applying”, “delivering,” “distributing,” “generating”, or “providing” positive-pressure, for example.

The term “reduced-pressure” (and “negative-pressure” or “hypobaric”) as used herein generally refers to a pressure less than a local ambient pressure, such as the ambient pressure in a local environment external to a sealed therapeutic environment (e.g., an internal volume). In most cases, this reduced-pressure will be less than the atmospheric pressure at which the patient is located. Alternatively, the reduced-pressure may be less than a hydrostatic pressure associated with tissue at the tissue site. Unless otherwise indicated, values of pressure stated herein are gauge pressures. References to increases in reduced-pressure typically refer to a decrease in absolute pressure, and decreases in reduced-pressure typically refer to an increase in absolute pressure. Additionally, the process of reducing pressure may be described illustratively herein as “applying”, “delivering,” “distributing,” “generating”, or “providing” reduced-pressure, for example.

The term “fluid” may refer to liquid, gas, air, or a combination thereof. The term “fluid seal,” or “seal,” means a seal adequate to maintain a pressure differential (e.g., positive-pressure or reduced-pressure) at a desired site given the particular pressure source or subsystem involved. Similarly, it may be convenient to describe certain features in terms of fluid “inlet” or “outlet” in such a frame of reference. However, the fluid path may also be reversed in some applications, such as by substituting a reduced-pressure source (negative or hypobaric pressure source) for a positive-pressure source, and this descriptive convention should not be construed as a limiting convention.

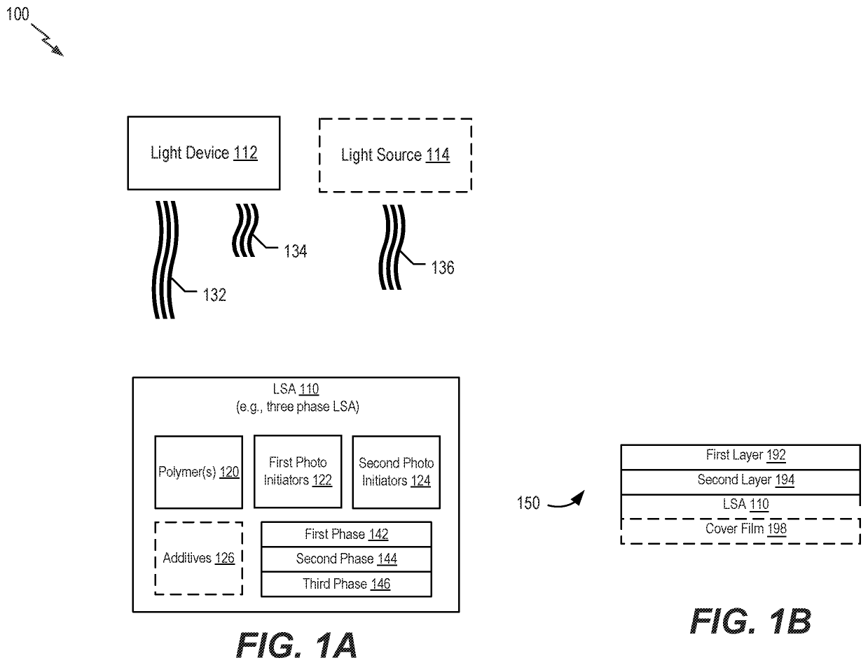

FIG. 1A illustrates a block diagram of an illustrative system 100 for activating a light switchable adhesive. System 100 includes a light switchable adhesive (LSA) 110 (e.g., a three or more phase light switchable adhesive), a light device 112, and an optional light source 114. System 100 is configured to activate the LSA 110 to transition or switch the LSA 110 between multiple phases, such as from a first phase 142 to a second phase 144, and from the second phase 144 to a third phase 146. For example, system 100 may apply light of different wavelengths to LSA 110 to activate photo initiators 122, 124 thereof to control a peel strength of the LSA 110. One such exemplary use of LSA 110 is as a repositionable light switchable adhesive for medical applications and devices.

The LSA 110 includes or correspond to a pressure sensitive adhesive. As illustrated in FIG. 1A, the LSA 110 includes one or more polymers 120, first photo initiators 122, and second photo initiators 124. The one or more polymers 120, the first photo initiators 122, and the second photo initiators 124 may include or correspond to a polymer composition. Although two types of photo initiators are illustrated in FIG. 1A, additional types of photo initiators may be included in other implementations. LSA 110 may include or correspond to a light switchable adhesive as described in International Patent Application Nos. PCT/US2018/049388 and PCT/US2018/060718, which are incorporated by reference herein to the extent they describes light switchable adhesives.

The one or more polymers 120 may include chains of one or more monomers (e.g., polymer chains) and free monomers. The one or more polymers 120 may include or correspond to an uncured or partially cured polymer composition and may be cured (or partially cured) responsive to receiving light from the light device 112, the light source 114, or both. In some implementations, the one or more polymers 120 is acrylic based, such as includes acrylate, urethane acrylate, and/or silicone acrylate based polymers and oligomers. The one or more polymers 120 may include or further include polyether, polyurethane, methacrylate, or a combination thereof.

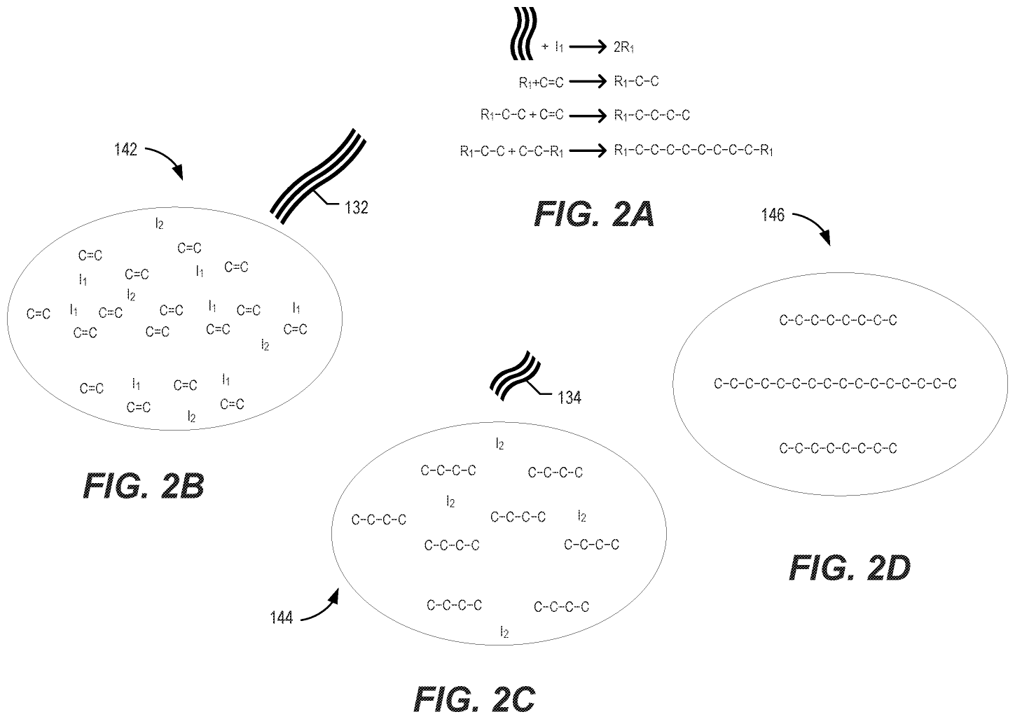

The first photo initiators 122 (e.g., first type photo initiators) are configured to cause cross-linking of monomers and/or polymer chains of the one or more polymers 120 to increase a degree of cross linking of the one or more polymers 120 or a degree of curing of the one or more polymers 120 responsive to receiving light of a particular wavelength, i.e., first light. For example, the first photo initiators 122 are configured to generate free radicals (e.g., first free radicals) responsive to receiving first light 132 from the light device 112. The free radicals are configured to active the one or more polymers 120 (e.g., monomers or polymer chains thereof) to increase bonding between the one or more polymers 120, such as increase polymer chain lengths, reduce free monomers, or both, as described further with reference to FIGS. 2A-2D.

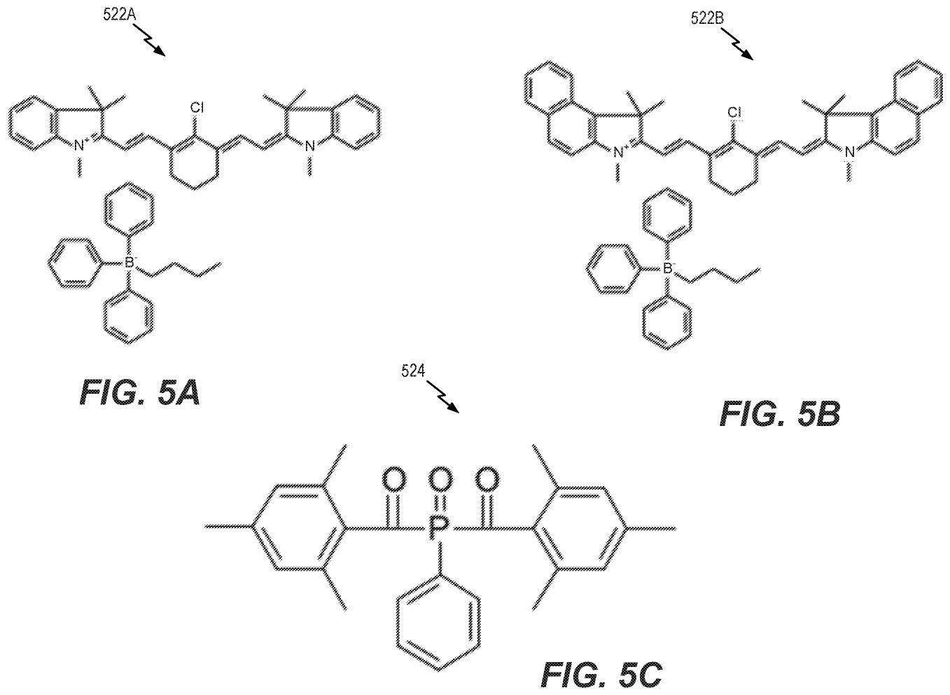

As an illustrative, non-limiting example, the first photo initiators 122 include infrared (IR) type photo initiators, i.e., photo initiators that are activated by light near or within the infrared spectrum. Additionally, or alternatively, the first photo initiators 122 include visible light type photo initiators or ultraviolet (UV) type photo initiators. Exemplary photo initiators are described further with reference to FIGS. 2A-2D and 5A-5C.

Similarly, second photo initiators 124 (e.g., second type photo initiators) are configured to cause cross-linking of monomers and/or polymer chains of the one or more polymers 120 to increase a degree of cross linking of the one or more polymers 120 or a degree of curing of the one or more polymers 120 responsive to receiving light of a particular wavelength, i.e., second light. For example, the second photo initiators 124 are configured to generate free radicals (e.g., second free radicals) responsive to receiving second light 134 from the light device 112 or third light 136 from the light source 114. The free radicals are configured to active the one or more polymers 120 (e.g., monomers or polymer chains thereof) to increase bonding between the one or more polymers 120, such as increase polymer chain lengths, reduce free monomers, or both.

In some implementations, the LSA 110 includes one or more additives 126. The additive 126 may include or correspond to additives to increase dissolution of the photo initiators 122, 124 in a particular polymer or to increase free radical production and/or curing. For example, DMAA (N,N-Dimethylacrylamide) and/or ketones can be used to increase solubility of photo initiators 122, 124 in acrylate resins. As another example, certain co-initiators, such as Borate V or Irgacure 184, may increase a speed of free radical cure.

Additionally, or alternatively, the additives 126 include a marking additive, such as an IR marking additive, a UV marking additive, or a visible light marking additive. Such marking additives may produce a visual indication, such as a color change, text, a symbol, etc., to indicate that light of a particular wavelength which may activate LSA 110 has been received or that a transition of phases or states has occurred.

In a particular implementation, the UV marking additive includes or corresponds to an ultraviolet absorber (UV absorber). A UV absorber is a molecule used in organic or synthetic materials to absorb UV radiation. The UV absorbers are configured to absorb at least a portion of UV radiation of the UV spectrum and produce a visual indication, such as a color change. For example, UVA absorbers are configured to absorb UVA radiation, i.e., electromagnetic radiation having wavelengths between 300 and 400 nm. Additionally, or alternatively, one or more other layers of a compound film 152 may include a UV marking additive or another additive, such as a visible light additive. For example, a first layer (e.g., 192, such as a light blocking layer or protective film) and/or a second layer (e.g., 194, such as a non-light blocking layer or adhesive layer) may include a marking additive. Such marking additives may produce a color change, produce text, produce a symbol, etc. to indicate light which may activate LSA 110 has been received.

Additionally, or alternatively, the reaction or reactions caused by a particular photo initiator may provide a visual indication. To illustrate, IR photo initiators may produce a color change upon receiving light and producing a free radical, upon the free radical altering a monomer/polymer, upon cross-linking or combining of monomers/polymers, or a combination thereof.

In some implementations, the first photo initiators 122 have a peak absorbance between 750 nanometers (nm) to 860 nm. In a particular implementation, the first photo initiators 122 have a peak absorbance of about 780 nm or about 815 nm. In some implementations, the second photo initiators 124 have a peak absorbance between 200 nanometers (nm) to 400 nm. In a particular implementation, the second photo initiators 124 have a peak absorbance of about 385 nm.

In some implementations, LSA 110 has a peel strength of less than 7 N/25 mm on stainless steel at an angle of 180 degrees in the first phase 142. In a particular implementation,

LSA 110 has a peel strength between 4 N/25 mm and 6 N/25 mm on stainless steel at an angle of 180 degrees in the first phase 142. In some such implementations, the light switchable adhesive has a peel strength of greater 8 N/25 mm on stainless steel at an angle of 180 degrees in the second phase 144. In a particular implementation, LSA 110 has a peel strength of greater than 15 N/25 mm on stainless steel at an angle of 180 degrees in the first phase 142. In another implementation, LSA 110 has a peel strength between 10 N/25 mm and 15 N/25 mm on stainless steel at 180 degrees in the first phase 142. Additionally, or alternatively, the light switchable adhesive has a peel strength of less than 7 N/25 mm on stainless steel at an angle of 180 degrees in a third phase 146. In a particular implementation, the LSA has a peel strength between 3 N/25 mm and 6 N/25 mm on stainless steel at an angle of 180 degrees in a third phase.

In some implementations, LSA 110 has an areal weight of 50 to 300 grams per square meter (gsm). In a particular implementation, LSA 110 has an areal weight of 100 to 250 grams per square meter (gsm). In some implementations, LSA 110 has a moisture vapor transfer rate (MVTR) of greater than 250 g/m² in the first phase 142, the second phase 144, or both. In a particular implementation, LSA 110 has a MVTR of 250 g/m² to 1000 g/m2 in the first phase 142, the second phase 144, or both. In other implementations, the LSA has a MVTR of greater than 1000 g/m² in the first phase 142, the second phase 144, or both.

Additionally, or alternatively, LSA 110 has a viscosity which produces 7 mm to 11 mm of cone penetration according to ISO 2137, alternatively referred to as 70 mm/10 to 110 mm/10, in the first phase 142, the second phase 144, or both, in some implementations. In a particular implementation, LSA 110 has a viscosity which produces 9 mm of cone penetration according to ISO 2137, alternatively referred to as 90 mm/10, in the first phase 142, the second phase 144, or both. To illustrate, the viscosity is measured by a penetrometer according to the standard Norfolk Island (NF) International Organization for Standardization (ISO) 2137, using a penetrometer PNR 12 Petrotest model with a total weight of the rod and cone attached thereto is 62.5 grams. Cone penetration of a sample (i.e., the LSA 110) in a container is determined at 25° C. by measuring the depth of penetration of the cone penetrometer into the sample after releasing the cone penetrometer and allowing the cone penetrometer to act for 5 seconds. The measured penetration depth is often multiplied by 10 and notated in “N” mm/10.

The light device 112 is configured to provide light to activate LSA 110 (i.e., photo initiators 122, 124, or both thereof) and cause LSA 110 to switch phases (e.g., 142-146), also referred to as states. Light device 112 may include or correspond to the Sun, ambient lighting, a dedicated light device, such as an infrared (IR) device, a visible light device, an ultraviolet (UV) device, a dual light device, or a combination thereof.

An exemplary IR device is configured to generate/emit IR light to activate LSA 110 (photo initiators thereof) and cause LSA 110 to switch phases (e.g., 142-146). For example, IR device includes or corresponds to a IR light source configured to generate IR light or electromagnetic radiation having a wavelength of 700 nanometers (nm)-1 millimeter (mm). In some implementations, IR device may include or correspond to a IR torch. For example, IR torch may include one or more LEDs configured to generate incoherent light in the IR spectrum. In a particular implementation, IR torch generates light in a particular sub spectrum of the IR spectrum, such as near-infrared (NIR or IR-A) or short-wavelength infrared (SWIR or IR-B).

In other implementations, the IR device may include or correspond to an IR Laser, such as a gas laser, a laser diode, a solid-state laser, an excimer laser, or a combination thereof. In some implementations, IR laser is configured to generate coherent light (e.g., a laser beam) having electromagnetic radiation of IR wavelengths. For example, IR laser is a IR-A laser (700-1400 nm), a IR-B laser (1400-3000 nm), or an IR-C laser (3000 nm-1 mm).

An exemplary UV device is configured to generate/emit UV light to activate LSA 110 (photo initiators thereof) and cause LSA 110 to switch phases (e.g., 142-146). For example, UV device includes or corresponds to a UV light source configured to generate light or electromagnetic radiation having a wavelength of 10-500 nanometers, such as UV light to blue light. In some implementations, UV device may include or correspond to a UV torch. For example, UV torch may include one or more LEDs configured to generate incoherent light in the UV spectrum. In a particular implementation, UV torch generates light in a particular subspectrum of the UV spectrum, such as UVA or UVC.

In other implementations, UV device may include or correspond to a UV Laser, such as a gas laser, a laser diode, a solid-state laser, an excimer laser, or a combination thereof. In some implementations, UV laser is configured to generate coherent light (e.g., a laser beam) having electromagnetic radiation of UV wavelengths. For example, UV laser is a UVA laser (315-400 nm), a UVB laser (280-315 nm), a UVC laser (100-280 nm), or an extreme UV laser (10-121 nm).

An exemplary visible light device is configured to generate/emit visible light to activate LSA 110 (photo initiators thereof) and cause LSA 110 to switch phases (e.g., 142-146). For example, visible device includes or corresponds to a visible light source configured to generate light or electromagnetic radiation having a wavelength of about 400-700 nanometers, such as violet light to red light. In some implementations, the visible light device may include or correspond to a visible light torch. For example, visible light torch may include one or more LEDs configured to generate incoherent light in the visible light spectrum. In a particular implementation, the visible light torch generates light in a particular subspectrum of the visible light spectrum, such as green light or orange light. In another implementations, the visible light torch generates light in various subspectrums of the visible light spectrum, such as violet, blue, green, yellow, etc. to generate “white” light.

Similarly, in some implementations, the light source 114 is configured to provide light to activate LSA 110 and cause LSA 110 to switch phases (e.g., 142-146). Light source 114 may include or correspond to the Sun, ambient lighting, a second dedicated light device, such as an ultraviolet (UV) device, or a combination thereof. In such implementations where the light source 114 is a second dedicated light device, the light source 114 may include or correspond to one or more components described with reference to the light device 112.

Although two photo initiators are illustrated in FIG. 1A, in other implementations additional photo initiators may be used. For example, LSA 110 includes third photo initiators configured to respond to the third light 136 or fourth light. The fourth light may be similar to the first light 132 or the second light 134 and may partially overlap the first light 132 or the second light 134. The third photo initiators can be used to augment or supplement (e.g., speed up) a particular transition, e.g., from the second phase 144 to the third phase 146 to facilitate removal, or the third photo initiators can be used to add an additional phase or state, such as a fourth phase or state. For example, the third photo initiators are configured to be activated responsive to receiving the first light 132, and the first photo initiators 122 and the third photo initiators (which are similar to the first photo initiators 122) transition the LSA from the first phase 142 to the second phase 144.

Alternatively, the third photo initiators can be used to add an additional phase or state. An additional phase or state can be used in attachment of the LSA 110, i.e., to get a larger increase in peel/bond strength, or in the removal of the LSA, i.e., to get a larger decrease in peel/bond strength. In some such implementations, the third photo initiator includes or corresponds to a visible light photo initiator. As illustrative, non-limiting examples, the visible light photo initiators include or correspond to H-Nu-Blue 660, H-Nu-Blue 660, or a combination thereof.

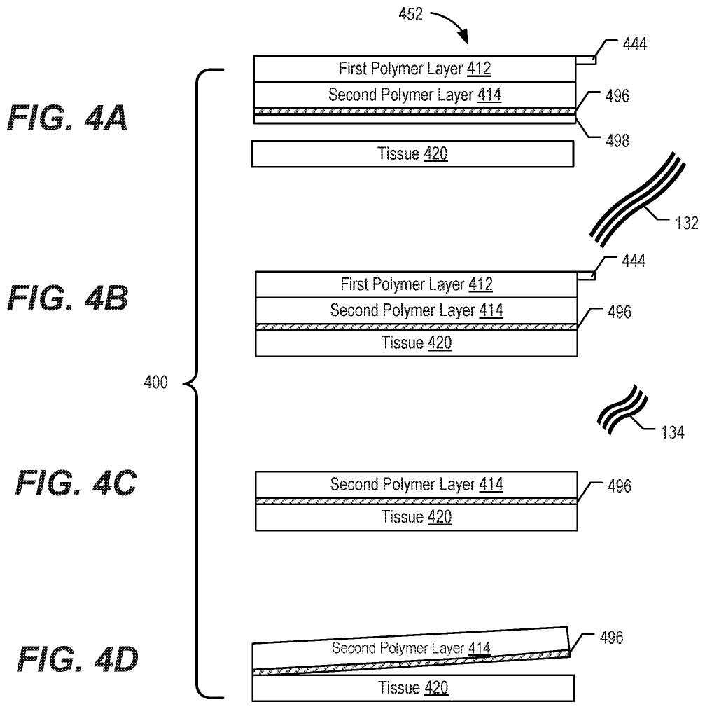

One particular use for LSA 110 is illustrated in FIG. 1B. Other examples of use and manufacture of an LSA, LSA 110, are described with reference to FIGS. 4A-4D, 6, and 7. Referring to FIG. 1B, a side view of a particular example of a compound film 152 including a light switchable adhesive is illustrated. In FIG. 1B, compound film 152 includes a first layer 192 (e.g., a protective film), a second layer 194 (e.g., an LSA host layer or adhesive layer), the LSA 110, and an optional cover film 198.

Layers 192, 194 may include polymer films. In a particular implementation, the layers 192, 194 have similar polymer materials or the same polymer material. For example, layers 192, 194 may both be polyurethane (PU) films, polyethylene (PE) films, etc. In light switchable adhesive related applications, one of layers 192, 194 is a light blocking film with respect to at least a first type of light (e.g., 132-136) and the other of layers 192, 194 is a non-light blocking film or light passing (e.g., transmitting) film with respect to a second type of light. In some implementations, the second layer 194 may include or correspond to a drape film. As described further herein, layers 192, 192 may include an impermeable or semi-permeable, elastomeric material, as an illustrative, non-limiting example. In some implementations, compound film 152 may be liquid/gas (e.g., moisture/vapor) impermeable or semi-permeable.

Compound film 152 is configured to be separable. In the example illustrated in FIG. 1B, the first layer 192 is a removable protective film, also referred to as a light blocking layer, and the second layer 194 is a non-light blocking layer (e.g., a light transmitting or passing layer). As compared to conventional compound films for conventional light switchable adhesives (e.g., two phase LSA), the compound films 152 described herein may include an additional protective film or light blocking layer corresponding to the different light, such as to filter or block the second light 134. Alternatively, the first layer 192 of the compound films 152 described herein may be configured to block light of multiple different spectrums, such as to filter or block the first light 132 and the second light 134. Additionally, in such implementations, the second layer 194 of the compound films 152 described herein may be configured to pass or transmit light of multiple different spectrums, such as the first light 132 and the second light 134. Accordingly, the compound film 152 supports blocking and receiving multiple types of light to control activation of the photo initiators 122, 124 of the LSA 110.

As illustrated in the example of FIG. 1B, the first layer 192 is in direct contact with the second layer 194, and the LSA 110 is in direct contact with the second layer 194. That is, compound film 152 does not include a handing or support layer or an adhesive layer between the first layer 192 and the second layer 194. In conventional light switchable adhesives, which are often thinner and less viscous, a support or handling layer is included in a compound film to provide handling of the compound film including the conventional light switchable adhesive during production, transportation, attachment, or a combination thereof. In a particular implementation, first layer 192 includes a tab (e.g., 444) to enable easy removal of the first layer 192 from the compound film 152. Tab may extend outwards and/or upwards from the compound film 152 to facilitate removal or first layer 192 from second layer 194.

First layer 192 is configured to be removed from second layer 194 while second layer 194 is bonded to a bond site, such as a tissue site (e.g., 422, 620). First layer 192 is configured to block or filter light of a particular wavelength associated with transitioning the LSA 110 from the first phase 142 to the second phase 144, and second layer 194 is configured to pass or transmit the light of the particular wavelength associated with transitioning the LSA 110 from the second phase 144 to the third phase 146. For example, the first layer 192 may be configured to block or filter UV light wavelengths, visible light wavelengths, or both and/or the second layer 194 may be configured to pass UV light wavelengths, visible light wavelengths, or both. To illustrate, the first layer 192 is configured to block or filter light having a wavelength between 10 nanometers and 500 nanometers and/or the second layer 194 is configured to pass light having a wavelength between 10 nanometers and 500 nanometers. In other implementations, the light which is blocked or filtered by the first layer 192 and/or passed by second layer 194 includes or corresponds to visible light, a portion of the visible light spectrum, UV light, a portion of the UV light spectrum, or a combination thereof. In a particular implementation, the first layer 192 is opaque and the second layer 194 is optically transparent.

In a particular implementation, first layer 192 and second layer 194 are configured to be permeable to air and water vapor, to enable tissue of tissue site to which the compound film 152 is bonded to “breathe.” First layer 192 and second layer 194 of compound film 152 may include an impermeable or semi-permeable, elastomeric material, as an illustrative, non-limiting example. In some implementations, first layer 192 and/or second layer 194 are liquid/gas (e.g., moisture/vapor) impermeable or semi-permeable. Additionally, or alternatively, first layer 192 and/or second layer 194 include or are elastomeric material. “Elastomeric” means having the properties of an elastomer. For example, elastomer generally refers to a polymeric material that may have rubber-like properties. More specifically, an elastomer may typically have ultimate elongations greater than or equal to 100% and a significant amount of resilience. The resilience of a material refers to the material's ability to recover from an elastic deformation. Elastomers that are relatively less resilient may also be used as these elastomers. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones.

In some implementations, second layer 194 is configured to diffuse light to LSA 110, such as light received from a top (e.g., when first layer 192 is removed) and/or a side of second layer 194. To illustrate, light (e.g., 132-136) received on a side of second layer 194 is scattered as it passes through second layer 194 to distribute the light to the LSA 110. Additionally, or alternatively, second layer 194 may be formed of a thin, clear, flexible, breathable material with a high refractive index. One exemplary material for the second layer 194 is polyurethane (PU).

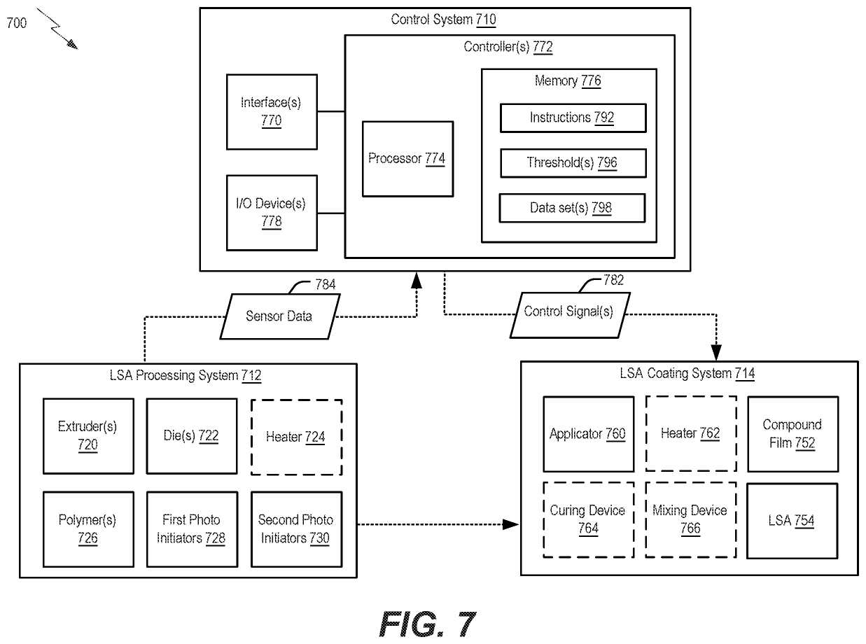

LSA 110 may be applied to or disposed on second layer 194 after compound film 152 is formed, as described with reference to FIG. 7. In some implementations, LSA 110 is a coating or a pattern of coatings, as described further herein. Alternatively, LSA 110 may be formed with one or more films of the compound film 152, such as co-extruded with second layer 194.

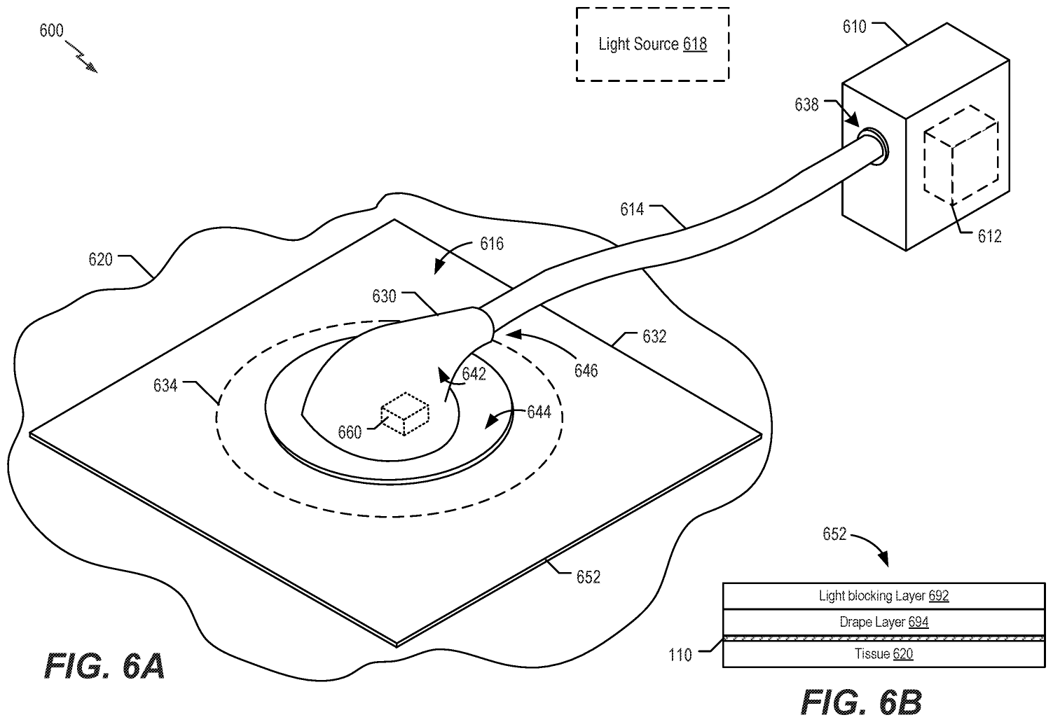

Compound film 152 may be configured to couple a bandage, a wound closure device, a dressing, and/or a drape, to provide a seal to create an enclosed space (e.g., an interior volume) corresponding to a tissue site. For example, compound film 152 may be configured to provide a fluid seal (i.e., provide a portion of fluid seal) between two components and/or two environments, such as between a sealed therapeutic environment and a local ambient environment. To illustrate, when coupled to a tissue site, compound film 152 is configured to maintain a pressure differential at the tissue site and/or keep fluids from permeating through the compound film 152, as described further with reference to FIG. 6A.

In some implementations, LSA 110 has or is configured to provide a bond strength (e.g., peel strength) for the compound film 152 of at least at or greater than, or substantially equal to any one of, or between two of: 3, 4, 5, 6, 8, 10, 12, 14, 16, 18, or 20 N, in the second phase 144. The bond may be formed by LSA 110 between second layer 194 and a bond site, such as target tissue of a tissue site. To illustrate, LSA 110 may have a bond strength as described above or may be applied such that compound film 152 has a bond strength as described above, such as when LSA 110 is patterned on compound film 152. In some implementations, the bond strength of the LSA 110 increases after application of LSA 110 to the bond site (e.g., tissue site). For example, the bond strength of the LSA 110 may achieve (e.g., reach) a maximum bond strength between 30 minutes to 2 hours after application. Additionally, or alternatively, LSA 110 has or is configured to provide a bond strength (e.g., peel strength) at least at or greater than, or substantially equal to any one of, or between two of: 0.3, 0.5, 1, 2, 3, 4, 5, 6, 8, or 10 N, in the third phase 146 after being exposed to second light 134. The peel strength values for LSA 110/compound film 152 or bond strength of LSA 110/compound film 152 are described in terms of a peel strength as measured by a 180 degree angle peel test on stainless steel at room temperature. The peel strength values for LSA 110/compound film 152 or bond strength of LSA 110/compound film 152 may be indicative of a bond strength with human tissue at a particular time, such as two hours after application of LSA 110/compound film 152.

The compound film 152 may be post processed as described further herein. For example, the compound film 152 may be perforated and/or may be coupled, bonded to, or compound with one or more additional films or layers. In some implementations, a light switchable adhesive is applied to compound film 152, as described further with reference to FIG. 7.

As described above, layers 192, 194 of compound film 152 are removable/separable, i.e. are designed to be removed from each other during operation of the compound film 152. An example of operation of a compound film including an LSA 110 is described further with reference to FIGS. 4A-4D.

In some implementations, a light switchable adhesive (e.g., three or more phase light switchable adhesive) includes one or more polymers; first photo initiators configured to cause the one or more polymers to cross-link responsive to receiving first light; and second photo initiators configured to cause the one or more polymers to cross-link responsive to receiving second light, the second photo initiators different from the first photo initiators.

In a particular implementation, the light switchable adhesive has at least three phases, each phase corresponding to a particular peel strength, and wherein the light switchable adhesive is configured to transition between a first two phases of the three phases based on activation of the first photo initiators and to transition between a second two phases of the three phases based on activation of the second photo initiators. Additionally or alternatively, the light switchable adhesive has a second peel strength in the second phase that is greater than a first peel strength of the light switchable adhesive in the first phase, and wherein a third peel strength of the light switchable adhesive in the third phase is less than the second peel strength.

In some implementations, the light switchable adhesive is included in a compound film. In a particular implementation, a compound film includes a first layer of a first polymer composition; a second layer of a second polymer composition, the second layer removeably coupled to the first layer; and the light switchable adhesive.

Thus, system 100 describes an improved light switchable adhesive. The light switchable adhesives described herein, such as LSA 110, include additional phases and may have a low tact/peel strength first phase for easier use. Therefore, LSA 110 is suitable for use in medical devices, such as bandages, drapes, dressings, and wound closures. LSA 110 enables medical devices to be repositionable, thereby avoiding or limiting waste from inadvertent contact and misplacement and avoiding or limiting tissue damage at tissue site and patient discomfort from repositioning. Accordingly, LSA 110 may enable improved wound care and therapy, thereby advancing patient comfort and confidence in the treatment.

FIG. 2A illustrates an illustrative chemical reaction process for an exemplary photo initiator, such as first photo initiators 122, second photo initiators 124, or both. FIG. 2A illustrates free-radical polymerization (FRP) reactions. FRP is a method of polymerization (chain-growth polymerization) by which a polymer forms or increases in chain length by the successive addition of free-radical building blocks. In LSA (e.g., 110), the free radicals are formed by the photo initiators receiving light. The free radical adds (nonradical) monomer units to an existing polymer chain, thereby growing the polymer chain.

Different types of photo-initiators generate different types of free radicals. As an illustrative example, a free radical is created by breaking an oxygen-oxygen bond (O—O bond) in a peroxide, such as benzoyl peroxide. Exemplary free radicals are capable of attacking an carbon-carbon double bound (C═C bond), such as an olefinic double bond of a vinyl monomer.

The free radical is then transferred to the monomer, forming an active center that can attach to other monomers. This step is called propagation, in which the free radical is propagated down the polymer chain. The final step is termination, in which two molecules containing free radicals react and form the final product.

Monomer molecules add onto the active site of a growing polymer chain one at a time. Growth of the polymer occurs at the active sites on the chain, which are typically at the chain-end(s). The addition of each monomer unit to the growing polymer chain regenerates the active site, enabling an additional monomer unit to be added. In chain growth polymerization, an activated species (initiator or active center) adds one monomer molecule to create a new active center (propagation step), which again adds another monomer molecule to create another active center and so on, so that the chain growth proceeds as a chemical chain reaction.

As illustrated in FIG. 2A, a photo initiator (I1) receives light and undergoes a chemical reaction to produce free radicals (R1). The free radicals (R1) bond with a carbon-carbon double bond (C═C) and in the process break one of the bonds of the carbon-carbon double bond. The newly formed carbon and free radical chain bonds with other chains including carbon-carbon double bonds. Chains having free radical ends bond to each other. Accordingly, the resulting reactions from the photo initiator receiving light increase a degree of cross-linking of the polymers of the LSA.

Referring to FIGS. 2B-2D, exemplary states of an LSA, such as LSA 110, are illustrated in representative chemical form. FIG. 2B illustrates a first state or first phase (e.g., 142) of the LSA that has a low level of cross-linking and unactivated photo initiators. As illustrated in FIG. 2B, the LSA includes many short molecules including a carbon-carbon double bond (C═C). These molecules represent monomers or short polymer chains. The LSA also includes multiple types of photo initiator molecules, (I1, I2).

FIG. 2C illustrates a second state or second phase (e.g., 144) of LSA that has a medium level of cross-linking and unactivated photo initiators of a second type. As illustrated in FIG. 2C, the LSA includes many medium molecules including a carbon-carbon single bond (C—C). These short molecules represent monomers or short polymer chains. The LSA includes only second photo initiator molecules (I2), as the first photo initiator molecules (I1) have been activated or used up when first light was applied to the LSA in the first state.

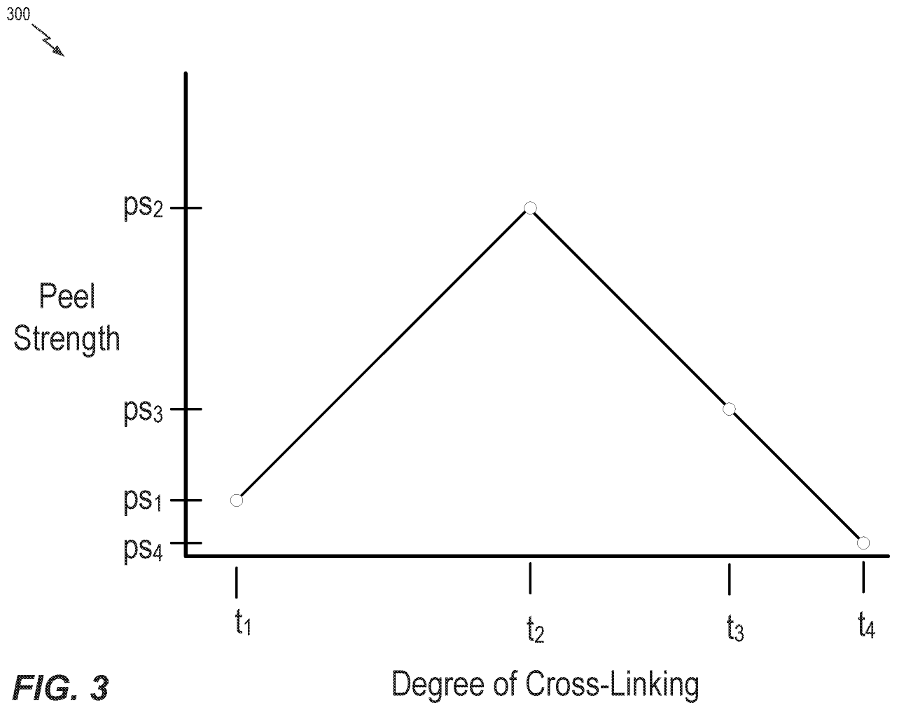

FIG. 2D illustrates a third state or third phase (e.g., 146) of LSA that has a high level of cross-linking and no unactivated photo initiators. As illustrated in FIG. 2D, the LSA includes a few longer polymer molecules including repeating monomers connected by a carbon-carbon single bond (C—C). The LSA also no longer includes any photo initiator molecules, (I1, I2), as the second photo initiator molecules (I2) have been activated or used up when second light was applied to the LSA in the second state. Corresponding peel strengths and curing/cross-linking levels for each state may be illustrated in FIG. 3.

Referring to FIG. 3, an exemplary graph 300 illustrating peel strength of an exemplary LSA and a degree of cross-linking of the LSA is shown. FIG. 3 illustrates a line graph illustrating peel strength values on a y-axis (vertical axis) and cross-linking values on an x-axis (horizontal axis) for transitions of an LSA, i.e., from a first state to a second state and from the second state to the third state. As an illustrative example, cross-linking values or a degree of cross-linking may include or correspond to an acrylate double bond conversion percentage. In FIG. 3, four exemplary peel strengths (ps1-ps4) and cross-linking degrees are illustrated at four corresponding times (t1-t4).

At a first time, t1, the LSA is uncured or partially cured, is in the first state (e.g., first phase 142), and has a first peel strength, ps1. From t1 to t2, the LSA undergoes a transition from the first state to the second state responsive to receiving first light. At a second time, t2, the LSA is partially cured, is in the second state (e.g., second phase 144), and has a second peel strength, ps2. From t2 to t3, the LSA undergoes a transition from the second state to a third state responsive to receiving second light. At a third time, t3, the LSA is fully cured, is in the third state (e.g., third phase 146), and has a third peel strength, ps3.

Alternatively, the third state or fully cured state has a peel strength that is lower than the first peel strength. As illustrated in FIG. 3, in some implementations the LSA can be cured to have a fourth peel strength in the third state. The fourth peel strength, ps4, corresponds to a fourth time, t4.

Graph 300 is an exemplary graph and the slopes (i.e. rate of change) of peel strength to degree of cross-linking is illustrative. The example slopes shown in FIG. 4A may be different from actual implementations of LSA and may differ based on which type of initiator is used. To illustrate, first photo initiators may induce more cross-linking more quickly than second photo initiators and thus, for example, a slope from t1 to t2 may be less than a slope from t2 to t3 or t4, as illustrated. As an example illustration, IR light may induce more cross-linking more quickly than UV or visible light and thus, for example, a slope from t1 to t2 may be greater than a slope from t2 to t3 or t4. Also, the example slopes are illustrated as linear or constant change, i.e., no acceleration or deceleration in the reaction for clarity. In actual examples, the slopes are likely to change (curves as opposed to lines) as the reactions are likely to slow when concentrations of reaction components decrease. Additionally, different formulations of LSA may have different slopes from each other. To illustrate, IR/UV formulations have different slopes from IR/Visible formulations.

FIGS. 4A-4D illustrates an example 400 of attaching and removing a compound film from a bond site, such as tissue 422. As illustrated in FIGS. 4A-4D, compound film 452 includes a first polymer layer 412, a second polymer layer 414, a LSA 496, and an adhesive cover film 498. Referring to FIGS. 4A and 4B, an example of attaching a compound film 452 to tissue 422 is shown. Compound film 452 may include or correspond to compound film 152. Layers 412, 414 may include or correspond to layers 192, 194, and LSA 496 may include or correspond to LSA 110. Tissue 422 may include or correspond to target tissue of a tissue site of a patient.

Although FIG. 4A illustrates that the compound film 452 includes adhesive cover film 498, the adhesive cover film 498 is optional and may not be included in some implementations. Adhesive cover film 498 (e.g., an adhesive cover layer) is positioned over or coupled to LSA 496 to protect LSA 496 from activation, i.e., receiving light and transitioning to between phases (e.g., 192-196), and from dust or contamination. Adhesive cover film 498 is configured to be removed prior to application of compound film 452 to tissue 422, and as such, adhesive cover film 498 has a lower peel strength or bond strength to the LSA 496 than a peel strength or bond strength between the LSA 496 and the second polymer layer 414 when the LSA 496 is in the first phase (e.g., 192). Adhesive cover film 498 may be formed of a thin, clear, flexible, breathable material with a high refractive index. One exemplary material for adhesive cover film 498 is polyurethane (PU).

FIG. 4A depicts a first state of compound film 452 prior to attachment to tissue 422 via LSA 496. FIG. 4B depicts a second state of compound film 452 after attachment of compound film 452 to tissue 422 via LSA 496. To attach compound film 452, the adhesive cover film 498 is removed from compound film 452 and the compound film 452 is attached to tissue 422. During attachment, compound film 452 may be repositioned, LSA 496 may be adjusted if unintended contact occurs, or a combination thereof. Additionally, LSA 496 may be worked into creases or surfaces of tissue 422 to create a strong, uniform bond. Once compound film 452 is in the desired position on tissue 422, first light 132 is applied to initiate curing/cross-linking and to transition the LSA 496 from the first phase (e.g., 142) to the second phase (e.g., 144). As illustrated in FIG. 4B, the first light 132 penetrates both the first polymer layer 412 and the second polymer layer 414. For example, the first polymer layer 412 and the second polymer layer 414 are transparent to IR wavelengths or a portion of IR wavelengths. In FIG. 4B, a bond strength between the LSA 496 and tissue 422 increases responsive to the first light 132, as described with reference to FIG. 3.

In other implementations, an additional polymer layer may be included to protect against wavelength used to cure LSA 496 and transition LSA 496 from the first phase to the second phase. For example, when visible light is used to transition LSA 496 from the first phase to the second phase, a third polymer layer may be coupled to the first polymer layer 412 (i.e., opposite the second polymer layer 414), and the third polymer layer is removed after attaching the compound film 452 to tissue 422 via LSA 496 but prior to application of the first light 132.

Referring to FIGS. 4C and 4D, an example of removing a compound film 452 from tissue 422 is shown. FIG. 4C depicts a third state of compound film 452 attached to tissue 422 via LSA 496. FIG. 4D depicts a fourth state of compound film 452 during removal of compound film 452.

Referring to FIG. 4C, the first polymer layer 412 is removed from the second polymer layer 414 by a patient or care provider, and second light 194 is applied to compound film 452 to initiate further curing/cross-linking and transition the LSA 496 from the second phase (e.g., 144) to the third phase (e.g., 146). In FIG. 4C, a bond strength between the LSA 496 and tissue 422 decreases responsive to the second light 134. Thus, a bond strength between the LSA 496 and tissue 422 in FIG. 4C after receiving the second light 134 is less than the bond strength between the LSA 496 and tissue 422 in FIG. 4B (after receiving the first light 132).

Referring to FIG. 4D, the second polymer layer 414, and optionally the LSA 496, is/are removed from the tissue 422 by a patient or care provider. In FIG. 4D, because the peel strength between the LSA 496 and the tissue 422 is less than a peel strength between the LSA 496 and the second polymer layer 414, the second polymer layer 414 and the LSA 496 detach from tissue 422. Additionally, because of the reduced peel strength of the LSA 496 in the third phase, the LSA 496 (and second polymer layer 414) may be removed from the tissue 422 without damage and pain.

In some implementations, a peel strength between the first polymer layer 412 and the second polymer layer 414 is between a peel strength of the LSA in the first phase and a peel strength of the LSA in the second phase, such as 4 N/25 mm to 8 N/25 mm. In a particular implementation, a peel strength between the first polymer layer 412 and the second polymer layer 414 is between 6 N/25 mm to 8 N/25 mm. Additionally, or alternatively, a peel strength between the second polymer layer 414 and the LSA 496 is greater than 4 N/25 mm. To illustrate, when LSA 496 is applied or disposed on the second polymer layer 414, the LSA 496 forms a bond with the second polymer layer 414 having a peel strength is greater than 3 N/25 mm in the first phase. In a particular implementation, a peel strength between the second polymer layer 414 and the LSA 496 is greater than 8 N/25 mm.

In some implementations, the LSA 496 is configured to have a peel strength of less than 6 N/25 mm between the LSA 496 and a tissue 422 prior to being cured, i.e., in the first phase. In a particular implementation, the LSA 496 is configured to generate a peel strength of 2 N/25 mm to 6 N/25 mm or of less than 4 N/25 mm between the LSA 496 and a tissue 422 prior to being cured. Additionally, or alternatively, the LSA 496 is configured to form a bond between the LSA 496 and a tissue 422 having a peel strength of less than 6 N/25 mm.

In some implementations, the LSA 496 is configured to generate a peel strength of greater than 6 N/25 mm between the LSA 496 and a tissue 422 within 2 hours after curing of the LSA 496 attached to the tissue 422. The tack level of the LSA 496 causes the LSA 496 to form a stronger bond with tissue 422 after application. Such a tack level allows for repositioning of the LSA 496 before the LSA 496 generates its maximum or operational bond strength. Adhesive cover film 498 may protect LSA 496 from dust and/or debris and enable easier handling to ensure that LSA 496 forms its maximum or operating bond. In a particular implementation, the LSA 496 is configured to generate a peel strength of 6 N/25 mm to 8 N/25 mm or of greater than 8 N/25 mm between the LSA 496 and a tissue 422 within 2 hours after curing of the LSA 496 attached to the tissue 422. Additionally, or alternatively, the LSA 496 is configured to form a bond between the LSA 496 and a tissue 422 having a peel strength of greater than 6 N/25 mm.

In some implementations, compound film 452 includes a tab 444 to facilitate removal of first polymer layer 412 from the compound film 452 (e.g., second polymer layer 414 thereof). Additionally or alternatively, other features may be added to control or influence peel strength, facilitate separation of layers, and/or protection of LSA, i.e., activations of photo-initiators thereof. Examples of such features include perforations in one or more layers of compound film 452, as described with reference to U.S. Prov. Pat. App. No. 62/816,351, which is incorporated by reference in its entirety herein. Another example feature includes patterns of LSA, such as described with reference to U.S. Prov. Pat. App. No. 62/816,351. Such features may be used alone or in combination with other features described herein.

FIGS. 5A-5C each illustrate a chemical formula for an exemplary photo initiator of an LSA, such as LSA 110 or LSA 496. Referring to FIG. 5A, a first chemical formula of a first photo initiator 522A is depicted. The first photo initiator 522A includes or corresponds to H-Nu-IR 780. The first photo initiator 522A may include or correspond to the first photo initiators 122.

Referring to FIG. 5B, a second chemical formula of a second photo initiator 522B is depicted. The second photo initiator second includes or corresponds to H-Nu-IR 815. The second photo initiator 522B may include or correspond to the first photo initiators 122. Thus, the first and second photo initiators 522A, 522B may be referred to as first type photo initiators 522A, 522B, and may be activated by similar wavelengths.

Referring to FIG. 5C, a third chemical formula is illustrated for a third photo initiator 524. The third photo initiator 524 is or includes Bis(2, 4, 6, trimethylbenzoyl)-phenylphosphineoxide. In a particular example, the third photo initiator 524 has a molecular weight of 418.5 and correspond to Irgacure 819. The third photo initiator 524 may include or correspond to the second photo initiators 124. The third photo initiator 524 may be referred to as a second type photo initiator.

In some implementations, first type photo initiators 522A, 522B have a concentration (e.g., weight percentage) of 0.05 to 3 percent of the total weight of the LSA (polymer composition. In a particular implementation, the first type photo initiators 522A, 522B have a concentration (e.g., weight percentage) of 0.1 to 1 percent of the total weight of the LSA (polymer composition).

Additionally, or alternatively, the second type photo initiators 524 have a concentration (e.g., weight percentage) of 0.5 to 8 percent of the total weight of the LSA (polymer composition. In a particular implementation, the second type photo initiators 524 have a concentration (e.g., weight percentage) of 1 to 4 percent of the total weight of the LSA (polymer composition).

In such implementations where a co-initiator or co-initiators are used, a first co-initiator for the first type photo initiators 522A, 522B may have a concentration (e.g., weight percentage) of 0.05 to 6 percent of the total weight of the LSA (polymer composition). In a particular implementation, the first co-initiator has a concentration (e.g., weight percentage) of 0.1 to 1 percent of the total weight of the LSA (polymer composition). As an illustrative, non-limiting example, a Borate V (from Spectra Group Limited) co-initiator may have a 1:1 ratio (mass concentration) with the first photo initiators.

Additionally, or alternatively, a second co-initiator for the second type photo initiators 524 may have a concentration (e.g., weight percentage) of 0.05 to 3 percent of the total weight of the LSA (polymer composition). In a particular implementation, the second co-initiator has a concentration (e.g., weight percentage) of 0.1 to 1 percent of the total weight of the LSA (polymer composition). As an illustrative, non-limiting example, an Irgacure 184 co-initiator may have a ratio (mass concentration) of 1:1 to 1:4 with the second type photo initiators 524.

In some implementation, a solvent may be added to the LSA (polymer composition) increase solubility of a photo initiator, a co-initiator, or both. For example, the LSA (polymer composition) has 2 to 3 percent by weight of DMAA and/or ketones, individually or in total.

FIG. 6A shows a perspective view of an illustrative system 600 (e.g., a therapy system) for providing wound therapy. System 600 may include a light switchable adhesive, such as LSA 110, a therapy device 610, a canister 612, a tube 614, a dressing 616, and a light source 618 (e.g., a UV device or a dual light device). As an illustrative example, system 600 includes LSA 110 as part of dressing 616 (e.g., drape 632 thereof). For example, LSA 110 is attached to a drape layer 694 of drape 632. The drape 632 includes a protective film 692 removably coupled to the drape layer 694 opposite the LSA 110, and the protective film 692 and drape layer 694 correspond to a compound film 652.

System 600 is configured to provide therapy (e.g., oxygen therapy, positive-pressure therapy, negative-pressure therapy, or a combination thereof) at a tissue site 620 associated with a target area of a patient. For example, dressing 616 may be in fluid communication with tissue site 620 and may be in fluid communication with therapy device 610 via tube 614. In some implementations, system 600 may include one or more components commercially available through and/or from KCI USA, Inc. of San Antonio, Tex., U.S.A., and/or its subsidiary and related companies (collectively, “KCI”).

Therapy device 610 (e.g., a treatment apparatus) is configured to provide therapy to tissue site 620 via tube 614 and dressing 616. For example, therapy device 610 may include a pressure source (e.g., a negative-pressure source, such as a pump, or a positive-pressure source, such as a pressurized oxygen container, an oxygen concentrator, or an oxygen collector) configured to be actuatable (and/or actuated) to apply pressure differential relative to ambient conditions to dressing 616. As illustrative, non-limiting examples, positive-pressure applied to a tissue site may typically ranges between 5 millimeters mercury (mm Hg) (667 pascals (Pa)) and 30 mm Hg (4.00 kilo (k) Pa). Common therapeutic ranges are between 10 mm Hg (1.33 kPa) and 25 mm Hg (3.33 kPa). As illustrative, non-limiting examples, reduced-pressure applied to a tissue site may typically ranges between −5 millimeters mercury (mm Hg) (−667 pascals (Pa)) and −500 mm Hg (−66.7 kilo (k) Pa). Common therapeutic ranges are between −75 mm Hg (−9.9 kPa) and −300 mm Hg (−39.9 kPa).

In some implementations, therapy device 610 may alternate between providing positive-pressure therapy and negative-pressure therapy to the dressing 616, may provide positive-pressure therapy to a first portion of the dressing 616 and negative-pressure therapy to a second portion of the dressing 616, may provide no positive or negative pressure, or a combination thereof. In some such implementations, the therapy device 610 can provide positive-pressure therapy and negative-pressure therapy to the dressing 616 at the same time (e.g., partially concurrently).

As illustrated in FIG. 6A, therapy device 610 includes canister 612 to receive fluid from tissue site 620 or to provide fluid to tissue site 620. Although canister 612 is illustrated as being internal to and/or integrated with therapy device 610, in other implementations, canister 612 is external to therapy device 610, as illustrated and described with reference to FIG. 1A.

Therapy device 610 may also include one or more other components, such as a sensor, a processing unit (e.g., a processor), an alarm indicator, a memory, a database, software, a display device, a user interface, a regulator, and/or another component, that further facilitate positive-pressure therapy. Additionally, or alternatively, therapy device 610 may be configured to receive fluid, exudate, and or the like via dressing 616 and tube 614. Therapy device 610 may include one or connectors, such as a representative connector 638. Connector 630 is configured to be coupled to tube 614. Additionally, or alternatively, therapy device 610 may include one or more sensors, such a pressure sensor (e.g., a pressure transducer). The one or more sensors may be configured to enable therapy device 610 to monitor and/or sense a pressure associated with tube 614 and/or dressing 616.

Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

In some implementations, tube 614 may include multiple lumens, such as a primary lumen (e.g., a positive-pressure/fluid lumen) for application of positive-pressure and/or communication of fluid, and one or more secondary lumens proximate to or around the primary lumen. The one or more secondary lumens (e.g., one or more ancillary/peripheral lumens) may be coupled to one or more sensors (of therapy device 610), coupled to one or more valves, as an illustrative, non-limiting example. Although tube 614 is described as a single tube, in other implementations, system 600 may include multiple tubes, such as multiple distinct tubes coupled to therapy device 610, dressing 616, or both.

As used herein, a “tube” broadly refers to a tube, pipe, hose, conduit, or other structure with one or more lumens adapted to convey fluid, exudate, and/or the like, between two ends. In some implementations, a tube may be an elongated, cylindrical structure with some flexibility; however, a tube is not limited to such a structure. Accordingly, tube may be understood to include a multiple geometries and rigidity. Tube 614 includes one or more lumens (e.g., one or more through conduits), such as a single lumen conduit or multiple single-lumen conduits. Tube 614 (e.g., a least one of the one or more lumens) is configured to enable fluid communication between therapy device 610 and dressing 616. For example, fluid(s) and/or exudate can be communicated between therapy device 610 and dressing 616, and/or one or more pressure differentials (e.g., positive-pressure, negative pressure, or both) can be applied by therapy device 610 to dressing 616. As an illustrative, non-limiting illustration, tube 614 is configured to deliver at least pressurized oxygen from therapy device 610 to dressing 616 to establish positive-pressure. Communication of fluid(s) and application of a pressure differential can occur separately and/or concurrently.

Dressing 616 includes a connector 630 (also referred to as a dressing connection pad or a pad), a drape 632, and a manifold 634 (also referred to as a distribution manifold or an insert). Drape 632 may be coupled to connector 630. To illustrate, drape 632 may be coupled to connector 630 via an adhesive, a separate adhesive drape over at least a portion of connector 630 and at least a portion of drape 632, or a combination thereof, as illustrative, non-limiting examples.

Drape 632 may be configured to couple dressing 616 at tissue site 620 and/or to provide a seal to create an enclosed space (e.g., an interior volume) corresponding to tissue site 620. For example, drape 632 may be configured to provide a fluid seal between two components and/or two environments, such as between a sealed therapeutic environment and a local ambient environment. To illustrate, when coupled to tissue site 620, drape 632 is configured to maintain a pressure differential (provided by a positive-pressure source or a negative-pressure source) at tissue site 620. Drape 632 may include a drape aperture that extends through drape 632 to enable fluid communication between device and target tissue. Drape 632 may be configured to be coupled to tissue site 620 via an adhesive, such as a medically acceptable, pressure-sensitive adhesive that extends about a periphery, a portion, or an entirety of drape 632. Additionally, or alternatively, drape 632 may be coupled to tissue site 620 via a double-sided drape tape, paste, hydrocolloid, hydrogel, and/or other sealing device or element, as illustrative, non-limiting examples.

Drape 632 may include an impermeable or semi-permeable, elastomeric material, as an illustrative, non-limiting example. In some implementations, drape 632 may be liquid/gas (e.g., moisture/vapor) impermeable or semi-permeable. Examples of elastomers may include, but are not limited to, natural rubbers, polyisoprene, styrene butadiene rubber, chloroprene rubber, polybutadiene, nitrile rubber, butyl rubber, ethylene propylene rubber, ethylene propylene diene monomer, chlorosulfonated polyethylene, polysulfide rubber, polyurethane (PU), EVA film, co-polyester, and silicones. In some implementations, drape 632 may include the “V.A.C.® Drape” commercially available from KCI. Additional, specific non-limiting examples of materials of drape 632 may include a silicone drape, 3M Tegaderm® drape, and a polyurethane (PU) drape such as one available from Avery Dennison Corporation of Pasadena, Calif. An additional, specific non-limiting example of a material of the drape 632 may include a 30 micrometers (μm) matt polyurethane film such as the Inspire™ 2317 manufactured by Exopack™ Advanced Coatings of Matthews, N.C.

Referring to FIG. 6B, drape 632 includes or comprises a compound film 652 coupled to tissue site 620 by LSA 110. The compound film 652 of drape 632 includes a protective film 692 and a drape layer 694. A layer or coating of LSA 110 is bonded to drape layer 694. Protective film 692 may include or correspond to first layer 192 or first polymer layer 412. Drape layer 694 may include or correspond to second layer 194 or second polymer layer 414. In some implementations, drape 632 includes LSA 110 on only a portion of the compound film 652, such as a portion of the compound film 652 about a periphery of the drape 632.

Referring to FIG. 6A, manifold 634 is configured to be positioned on and/or near tissue site 620, and may be secured at the tissue site 620, such as secured by drape 632. The term “manifold” as used herein generally refers to a substance or structure that may be provided to assist in applying a pressure differential (e.g., positive-pressure differential) to, delivering fluids to, or removing fluids and/or exudate from a tissue site and/or target tissue. The manifold typically includes a plurality of flow channels or pathways that distribute fluids provided to and removed from the tissue site. In an illustrative implementation, the flow channels or pathways are interconnected to improve distribution of fluids provided to or removed from the tissue site. Manifold 634 may be a biocompatible material that may be capable of being placed in contact with the tissue site and distributing positive and/or negative-pressure to the tissue site. Manifold 634 may include, without limitation, devices that have structural elements arranged to form flow channels, such as foam, cellular foam, open-cell foam, porous tissue collections, liquids, gels, and/or a foam that includes, or cures to include, flow channels, as illustrative, non-limiting examples. Additionally, or alternatively, manifold may include polyethylene, a polyolefin, a polyether, polyurethane, a co-polyester, a copolymer thereof, a combination thereof, or a blend thereof.

In some implementations, manifold 634 is porous and may be made from foam, gauze, felted mat, or other material suited to a particular biological application. In a particular implementation, manifold 634 may be a porous foam and may include a plurality of interconnected cells or pores that act as flow channels. The foam (e.g., foam material) may be either hydrophobic or hydrophilic. As an illustrative, non-limiting example, the porous foam may be a polyurethane, open-cell, reticulated foam such as GranuFoam® material manufactured by Kinetic Concepts, Incorporated of San Antonio, Tex.

In some implementations, manifold 634 is also used to distribute fluids such as medications, antibacterials, growth factors, and other solutions to the tissue site. Other layers may be included in or on manifold 634, such as absorptive materials, wicking materials, hydrophobic materials, and hydrophilic materials. In an implementation in which the manifold 634 includes a hydrophilic material, manifold 634 may be configured to wick fluid away from tissue site 620 and to distribute positive-pressure to tissue site 620. The wicking properties of manifold 634 may draw fluid away from the tissue site 620 by capillary flow or other wicking mechanisms. An illustrative, non-limiting example of a hydrophilic foam is a polyvinyl alcohol, open-cell foam such as V.A.C. WhiteFoam® dressing available from Kinetic Concepts, Inc. of San Antonio, Tex. Other hydrophilic foams may include those made from polyether and/or foams that have been treated or coated to provide hydrophilicity.

In some implementations, manifold 634 is constructed from bioresorbable materials that do not have to be removed from tissue site 620 following use of the system 600. Suitable bioresorbable materials may include, without limitation, a polymeric blend of polylactic acid (PLA) and polyglycolic acid (PGA). The polymeric blend may also include without limitation polycarbonates, polyfumarates, and capralactones. Manifold 634 may further serve as a scaffold for new cell-growth, or a scaffold material may be used in conjunction with manifold 634 to promote cell-growth. A scaffold may be a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials. Although a manifold 634 is illustrated in FIG. 6A, in other implementations, dressing 616 does not include manifold 634. In such implementations, drape 632 of dressing 616 is coupled to connector 630.

Connector 630 includes a body 642 (e.g., a housing) and a base 644, and is configured to be coupled to tube 614 via an interface 646 (e.g., a port). Base 644 is configured to be coupled to dressing 616. For example, base 644 may be coupled, such as via an adhesive, to drape 632 and/or manifold 634. In some implementations, base 644 comprises a flange that is coupled to an end of body 642 and/or is integrally formed with body 642. Connector 630, such as body 642, base 644, interface 646, or a combination thereof, may be made of rigid material and/or a semi-rigid material. In a non-limiting example, connector 630 may be made from a plasticized polyvinyl chloride (PVC), polyurethane, cyclic olefin copolymer elastomer, thermoplastic elastomer, poly acrylic, silicone polymer, or polyether block amide copolymer. In some implementations, connector 630 is formed of a semi-rigid material that is configured to expand when under a force, such as positive-pressure greater than or equal to a particular amount of pressure. Additionally or alternatively, connector 630 may be formed of a semi-rigid material that is configured to collapse when under a force, such as reduced-pressure less than or equal to a threshold pressure.

Body 642 includes one or more channels or one or more conduits that extend from and/or are coupled to interface 646. To illustrate, body 642 may include a primary channel configured to be coupled in fluid communication with a primary lumen (e.g., 621) of tube 614. The primary channel may be coupled to a cavity (e.g., a tissue cavity partially defined by body 642) having an aperture open towards manifold 634 (and/or towards tissue site 620). For example, the primary channel may include a first opening associated with interface 646 and a second opening (distinct from the aperture of the cavity) associated with the cavity. Thus, the primary channel may define a through channel of body 642 to enable fluid communication between interface 646 and tissue site 620.

Body 642 includes a channel (e.g., a through channel) having a first aperture open opposite dressing 616 and a second aperture open towards dressing 616. For example, the first aperture is located on an outer surface side (e.g., an ambient environment surface) of connector 630 and the second aperture is located on an inner surface side (e.g., a tissue facing side) of connector 630. The second aperture is configured to be coupled to one or more lumens of tube 614, such as coupled via the cavity. Illustrative, non-limiting examples of commercially available connectors include a “V.A.C. T.R.A.C.® Pad,” or “Sensa T.R.A.C.® Pad” available from Kinetic Concepts, Inc. (KCI) of San Antonio, Tex.

In some implementations. dressing 616 further includes a bandage and/or a wound closure device 660. For example, a bandage may be placed over a wound to protect the wound and a wound closure device 660 may be placed proximate to a wound to provide a force to maintain tissue in fixed position to promote wound closure. Each of the bandage and/or a wound closure device 660 may include compound film 652.

Light source 618 is configured to provide light to activate LSA 110 (e.g., one or more type of photo initiators thereof) and cause LSA 110 to switch phases or states. Light source 618 may include or correspond to the Sun, ambient lighting, a dedicated light device, such as an IR torch, an UV torch device, visible light torch, a dual light torch, or a combination thereof. In a particular implementation, light source 618 is included in or integrated with therapy device 610. In some such implementations, light source 618 provides light to drape 632 via tube 614.

During operation of system 600, dressing 616 is coupled to tissue site 620 over a wound. Additionally, dressing 616 is coupled to device 610 via tube 614. In some implementations, prior to coupling the dressing 616 to the tissue site 620, a bandage or a wound closure device 660 is coupled to tissue site 620 proximate to a wound. The dressing 616 is then coupled over the bandage or wound closure device 660. One or more of the dressing 616 or over the bandage or wound closure device 660 is coupled to tissue site 620 site via compound film 652. To illustrate, LSA 110 of the compound film 652 bonds the dressing 616, the bandage or wound closure device 660, or both to the tissue site 620 responsive to pressure. In a particular implementation when the compound film 652 is included in or corresponds to drape 632, and the compound film 652 may seal a portion of tissue site 620, such as an interior volume of dressing 616. First light (e.g., 132) is applied to the LSA 110 to partially cure the light switchable adhesive 110 and transition the LSA 110 from a first phase (e.g., 142) to a second phase (e.g., 144), such as from a low tact/peel strength state to a high tact/peel strength state.

A pressure differential, such as positive-pressure, can be generated and/or applied to dressing 616 (e.g., the interior volume of dressing 616) by a pressure source associated with device 610. When positive-pressure is generated and/or applied to dressing 616, fluid or medication from device 610, such as from canister 612, may be transported to dressing 616. Furthermore, in some implementations, reduced-pressure can be applied to dressing 616 (e.g., the interior volume of dressing 616 or a second interior volume of the dressing 616) by a reduced-pressure source associated with device 610. When reduced-pressure is applied to dressing 616 (e.g., when vacuum pressure is generated, fluid, exudate, or other material within dressing 616 may be transported to canister 612 of device 610.

After operation, such as completion of therapy, system 600 may be disconnected and components thereof removed from tissue site 620. For example, protective film 692 of compound film 652 may be removed from drape layer 694 exposing LSA 110 thereof to second light, such as ambient light or light from a dedicated light device (e.g., 112 or 114). The LSA 110 disposed on drape layer 694 may transition from the second phase (e.g., 144) to the third phase (e.g., 146). To illustrate, the LSA 110 transitions from the high tack/peel strength phase to a second low tack/peel strength phase by curing further (further increasing in cross-linking). Accordingly, drape 632, and thus dressing 616, can be easily removed from tissue site 620. In some implementations where a bandage/wound closure device 660 is used and where the bandage/wound closure device 660 includes a compound film 652, the LSA 110 can be activated by second light (e.g., 134 or 136), such as UV light. To illustrate, the protective film 692 of the compound film 652 of the bandage/wound closure device 660 may be removed from drape layer 694 exposing LSA 110 to the second light. Similarly, the bandage/wound closure device 660 can be easily removed from tissue site 620.

Thus, dressing 616, bandage/wound closure device 660, or both, can be adhered to a patient with a light switchable adhesive in a low tack phase to be painlessly and easily repositioned. Accordingly, the light switchable adhesive enables easier use and less waste, as compared to conventional light switchable adhesives with two phases or states.

Referring to FIG. 7, a block diagram of a manufacturing system, system 700, for making LSA and components (e.g., compound films) including LSA, such as a coating thereof. In the example illustrated in FIG. 7, system 700 includes a control system 710, a LSA processing system 712, and a LSA coating system 714. Control system 710 is configured to control one or more of systems 712 and 714, as described further herein.

LSA processing system 712 includes one or more extruders 720, one or more dies 722, and optionally includes one or more heaters (e.g., heating devices). LSA processing system 712 may include or correspond to extruder 214 and die 216 of FIG. 2. LSA processing system 712 is configured to generate LSA 754 from one or more polymers 726, first photo initiators 728, and second photo initiators 730. For example, LSA processing system 712 may be configured to generate LSA 754, such as three phase, duel electromagnetic radiation LSA. LSA processing system 712 may include or correspond to an extrusion film system. For example, LSA processing system 712 receives or generates pellets or resin of one or more polymers 726 or receives a polymer composition (e.g., polymer composite) including one or more polymers 726, and LSA processing system 712 produces extrudate of a polymer material based on the received polymer material. The extrudate may have the form of or may be formed into a film of polymer material (i.e., a polymer film of a polymer composition). The extrudate may correspond to LSA 754 or may be post processed into LSA 754. As an illustrative example, LSA processing system 712 may include or correspond to a melt-compounding system or a melt-blend combiner.

LSA coating system 714 is configured to apply LSA 754 to or form a coating of LSA on a film, such as a compound film 752. For example, LSA coating system 714 is configured to apply or selectively apply LSA 754 to compound film 752. LSA coating system 714 includes an applicator 760 and LSA 754. Applicator 760 may be configured to apply the LSA 754 to compound film 752 in a pattern, i.e., apply a pattern of LSA 754. For example, applicator 760 selectively applies the LSA 754 according to patterns 502-518, or applicator 760 applied a coating of LSA 754 and a removal device (e.g., a blade, a scraper, a wiper, a roller, etc.) selectively removes a portion of the coating. In some implementations, the applicator 760 is a die (e.g., a slot die), a roller, a patterned roller, a spray nozzle, etc.

LSA coating system 714 may optionally include one or more heaters 762, curing devices 764, mixing devices 766, or a combination thereof. The one or more heaters 762 and mixing devices 766 may be configured to heat and mix LSA 754 prior to application and/or delivery to applicator 760. The one or more curing device 764 may be configured to apply heat or light to the LSA 754 after application by the applicator 760. The compound film 752 may include or correspond to compound film 152, compound film 452, or compound film 652, and may be received from a film generation/lamination system.

Although listed as separate systems, systems 712 and 714 may be incorporated into a single system. For example, LSA processing system 712 and LSA coating system 714 may be incorporated into a single system. Additionally, system 700 may include one or more other systems, such as a film or compound film generation system, a cover film lamination system, a post-processing system, a packing system, a sterilization system, or a combination thereof. The post-processing system may be configured to cut and/or form the compound film 752 into shapes and add features to the compound film 752. For example, the post-processing system may modify the compound film to add tabs (e.g., 444).

Control system 710 includes one or more interfaces 770, one or more controllers, such as a representative controller 772, and one or more input/output (I/O) devices 778. Interfaces 770 may include a network interface and/or a device interface configured to be communicatively coupled to one or more other devices, such as LSA processing system 712 or LSA coating system 714. For example, interfaces 770 may include a transmitter, a receiver, or a combination thereof (e.g., a transceiver), and may enable wired communication, wireless communication, or a combination thereof. Although control system 710 is described as a single electronic device, in other implementations system 700 includes multiple electronic devices. In such implementations, such as a distributed control system, the multiple electronic devices each control a sub-system of system 700, such as LSA processing system 712 or LSA coating system 714.

The one or more controllers (e.g., controller 772) includes one or more processors and one or more memories, such as representative processor 774 and memory 776. The one or more controllers may include or correspond to an LSA processing controller, an LSA application controller, or a combination thereof. For example, LSA processing controller (e.g., processor 774) may be configured to generate and/or communicate one or more control signals 782 to LSA processing system 712. LSA processing controller may be configured to control (or regulate) an environment, such as an air quality, temperature, and/or pressure, within LSA processing system 712 (e.g., an extruder thereof) and/or delivery/injection of materials into LSA processing system 712. For example, LSA processing controller may be configured to generate and/or communicate one or more control signals 782, such as environment control signals, ingredient delivery control signals, or a combination thereof, to LSA processing system 712.

LSA application controller may be configured to control (or regulate) an environment, such as a temperature (e.g., heat) and/or pressure of LSA 754, applicator 760, or both, within LSA coating system 714 (e.g., applicator 760 thereof) and/or delivery/injection of LSA 754 into LSA coating system 714 (e.g., applicator 760 thereof). For example, application controller may be configured to generate and/or communicate one or more control signals 782, such as environment control signals, ingredient delivery control signals, or a combination thereof, to LSA coating system 714.

Memory 776, such as a non-transitory computer-readable storage medium, may include volatile memory devices (e.g., random access memory (RAM) devices), nonvolatile memory devices (e.g., read-only memory (ROM) devices, programmable read-only memory, and flash memory), or both. Memory 776 may be configured to store instructions 792, one or more thresholds 796, and one or more data sets 798. Instructions 792 (e.g., control logic) may be configured to, when executed by the one or more processors 774, cause the processor(s) 774 to perform operations as described further here. For example, the one or more processors 774 may perform operations as described with reference to FIGS. 1A, 1B, 2A-2D, 3, 4A-4D, 6, and 7. The one or more thresholds 796 and one or more data sets 798 may be configured to cause the processor(s) 774 to generate control signals. For example, the processors 774 may generate and send control signals responsive to receiving sensor data from one or more of system 712 and 714, such as exemplary sensor data 784 from LSA coating system 714. The temperature or ingredient flow rate can be adjusted based on comparing sensor data to one or more thresholds 796, one or more data sets 798, or a combination thereof.

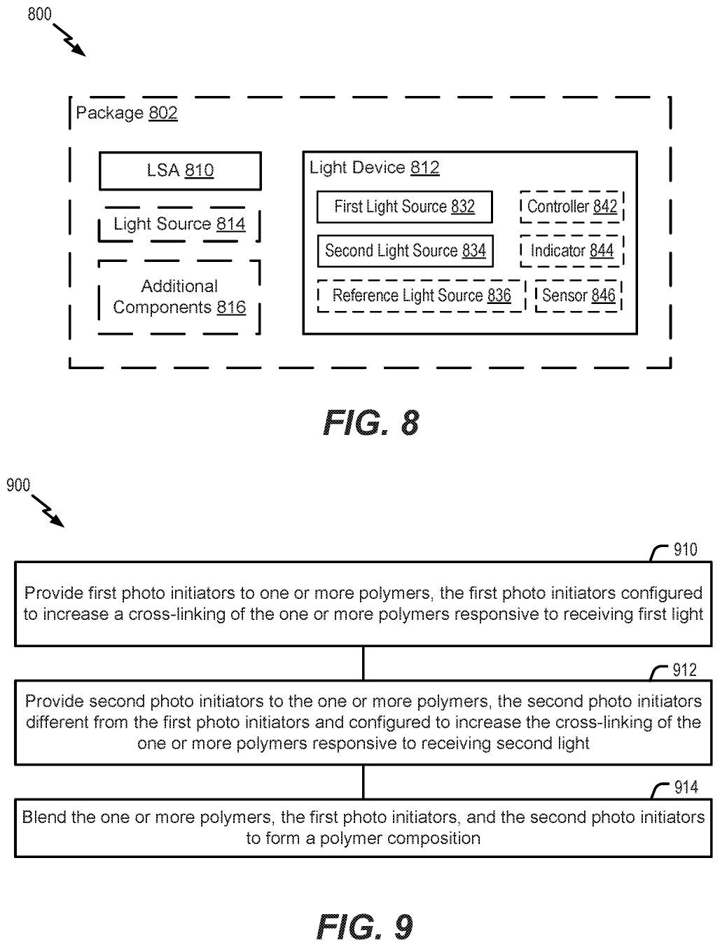

In some implementations, processor 774 may include or correspond to a microcontroller/microprocessor, a central processing unit (CPU), a field-programmable gate array (FPGA) device, an application-specific integrated circuits (ASIC), another hardware device, a firmware device, or any combination thereof. Processor 774 may be configured to execute instructions 792 to initiate or perform one or more operations described with reference to FIG. 1A, FIG. 2, and/or one more operations of the methods of FIGS. 8 and 9.

The one or more I/O devices 778 may include a mouse, a keyboard, a display device, the camera, other I/O devices, or a combination thereof. In some implementations, the processor(s) 774 generate and send control signals responsive to receiving one or more user inputs via the one or more I/O devices 778.

Control system 710 may include or correspond to an electronic device such as a communications device, a mobile phone, a cellular phone, a satellite phone, a computer, a tablet, a portable computer, a display device, a media player, or a desktop computer. Additionally, or alternatively, the control system 710 may include a personal digital assistant (PDA), a monitor, a computer monitor, a television, any other device that includes a processor or that stores or retrieves data or computer instructions, or a combination thereof.

During operation of system 700, LSA processing system 712 generates LSA 754. For example, LSA processing system 712 generates LSA 754 by mixing one polymer(s) 726 and photo initiators 728, 730. To illustrate, controller 772 may send one or more control signals 782 to LSA processing system 712. The control signals 782 may include signals configured to cause LSA processing system 712 to mix or blend polymer(s) 726 (e.g., resin or pellets thereof), and photo initiators 728, 730 to form a polymer composition or blend in extruder 720. To illustrate, control system 710 may send one or more signals 782 (e.g., environment control signals) to LSA processing system 712 to adjust conditions (e.g., heat, pressure, air quality) of the LSA processing system 712 or conditions (e.g., viscosity, temperature, etc.) of the polymer composition. Additionally or alternatively, control system 710 may send one or more control signals 782 (e.g., ingredient delivery control signals) to LSA processing system 712 to adjust rates and or amounts of polymer(s) 726, photo initiators 728, 730, one or more additives (e.g., 126), or a combination thereof.

In some implementations, heater 724 provides heat to extruder 720 or to polymer(s) 726 prior to delivery to the extruder 720. The polymer composition or blend is extruded by extruder 720 via a die 722 to form extrudate. The extrudate may include or correspond to the LSA 754.

After formation of LSA 754, the LSA 754 is provided to LSA coating system 714 and LSA coating system 714 applies or forms a coating of LSA 754 on the compound film 752. For example, LSA coating system 714 may form a coating or film of LSA 754 on the compound film 752 via selective application. To illustrate, control system 710 may send or more control signals to control delivery (e.g., application) of LSA 754 to applicator 760 of LSA coating system 714, LSA 754 to compound film 752 via applicator 760, or both. In other implementations, LSA coating system 714 forms the LSA 754 on the compound film 752 via selective removal. To illustrate, control system 710 may send or more control signals to control removal (e.g., scraping or removing) of LSA 754 from compound film 752.

In some implementations, LSA coating system 714 may receive control signals 782 to control a heater 762 and/or a mixing device 766 to heat and/or mix the LSA 754 prior to delivery of LSA 754 to applicator 760. Additionally, LSA coating system 714 may receive control signals 782 to control a curing device 764 to cure the LSA 754 applied to the compound film 752.

Thus, system 700 of FIG. 7 produces light switchable adhesives with three or more states or phases. Accordingly, the present disclosure enables manufacturing of a three or more phase LSA and inclusion of such LSA into products.

Referring to FIG. 8, a kit 800 for medical devices, such as a component of system 600, is illustrated. Kit 800 includes LSA 810, a light device 812, or both. The LSA 810 may include or correspond to LSA 110 or LSA 496. The light device 812 may include or correspond to light device 112, light source 114, light source 618, or a combination thereof.

In some implementations, the light device 812 (i.e., a dual light device) includes a first light source 832 and a second light source 834. The first light source 832 is configured to emit first light (e.g., 132) to activate first photo initiators (e.g., 122) of LSA 810, and the second light source 834 is configured to emit second light (e.g., 134, 136) to activate second photo initiators (e.g., 124) of LSA 810. For example, the first light source may include or correspond to light device 112, and the second light source may include or correspond to light source 114.

In some implementations, the light device 812 further includes a reference light source 836 configured to emit reference light. The reference light source 836 may provide an indication of one or more distances. For example, the reference light source 836 may produce a particular shape or object (e.g., be in focus) with the reference light when the reference light source 836 is at a particular distance that corresponds to providing a particular light from one of the light sources 832, 834. As an illustrative example, the reference light source 836 includes two white light sources angled relative to one another such that the reference light emitted from each white light source converges at a particular distance. Accordingly, an operator of the light device 812 can determine a distance for application of first light, second light or both.

In a particular implementation, the light device 812 further includes a controller 842, an indicator 844, and a sensor 846 (e.g., light or distance sensor). The controller 842 is configured to receive sensor data from the sensor 846 and to provide an indication via the indicator 844 that indicates a distance between the light device 812 and a surface. The indication may be visual, auditory, or haptic based, and the indication may indicate a particular distance (e.g., show an actual distance) or may indicate when the determined distance is within a range of distance.

In some implementations, LSA 810 is included in or on a compound film, such as compound film 150, compound film 452, a drape 632, compound film 752, etc. Additionally, or alternatively, LSA 810 is included in a container (e.g., a tube of LSA) and kit 800 includes an applicator.

In some implementations, kit 800 further includes a light source 814, one or more additional components 816, or a combination thereof. The light source 814 may include or correspond to a light source 114 or light source 618. The one or more additional components 816 may include or correspond to an LSA applicator, gloves, antiseptic, medical adhesive, and/or other components.

In some implementations, kit 800 may include a package 802. For example, package 802 may include a box, a bag, a container, or the like. Package 802 may include the LSA 810 and/or the light device 812. In some implementations, package 802 may further include the light source 814, the one or more additional components 816, or a combination thereof. Additionally, or alternatively, package 802 may include a packaging medium (e.g., packaging material), such as foam, paper, or the like. Thus, FIG. 8 describes kit 800 for a medical device that is secured to a tissue site or together by LSA (e.g., three phase LSA).

FIG. 9 illustrates a method 900 of manufacturing light switchable adhesive. The method 900 may be performed at or by system 700 (e.g., systems 712 and/or 714 thereof). Method 900 includes providing first photo initiators to one or more polymers, the first photo initiators configured to increase a cross-linking of the one or more polymers responsive to receiving first light, at 910. For example, the first photo initiators may include or correspond to first photo initiators 122, first photo initiators 522a, or first photo initiators 522b, and the one or more polymers may include or correspond to one or more polymers 120. The first light may include or correspond to first light 132 or third light 136.

Method 900 also includes providing second photo initiators to the one or more polymers, the second photo initiators different from the first photo initiators and configured to increase the cross-linking of the one or more polymers responsive to receiving second light, at 912. For example, the second photo initiators may include or correspond to second photo initiators 124 or second photo initiators 524, and the second light may include or correspond to second light 134 or third light 136.

Method 900 further includes blending the one or more polymers, the first photo initiators, and the second photo initiators to form a polymer composition, at 914. For example, the polymer composition may include or correspond to a light switchable adhesive, such as LSA 110, LSA 754, or LSA 810. To illustrate, a melt-blend combiner (e.g., an extruder or extrusion system), melt blends the ingredients 120-124 together to form a polymer composition that corresponds to LSA.

In some implementations, method 900 further comprises blending one or more additives to form the polymer composition. For example, the additives may include or correspond to additives 126, such as co-initiators, solvents, or both. Additionally, or alternatively, the method further includes applying, such as by LSA coating system 714, the polymer composition to a film, such as compound film 150.

Thus, method 900 describes method of manufacturing a light switchable adhesive. The light switchable adhesive (e.g., a three or more phase light switchable adhesive) enables medical devices to be repositionable and be more resistant to inadvertent contact as compared to conventional light switchable adhesives, thereby increasing usability and reducing waste and patient discomfort. Accordingly, the light switchable adhesives described herein may enable improved wound care and therapy, thereby advancing patient comfort and confidence in the treatment.

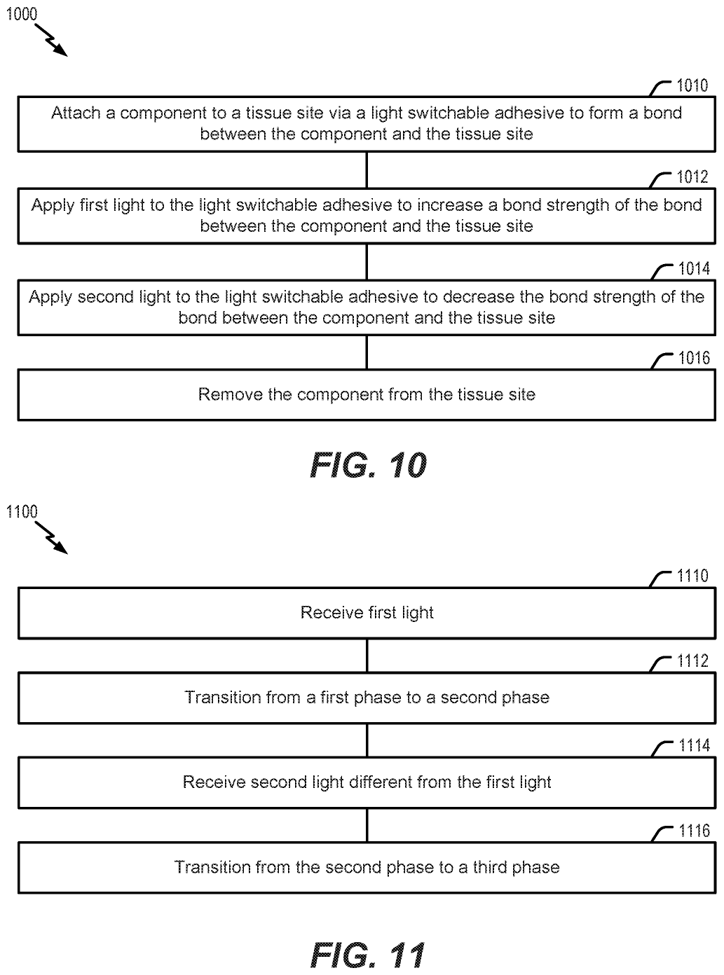

FIG. 10 illustrates a method 1000 of using a light switchable adhesive to attach a component to a tissue site. The method 1000 may be performed by a patient or care provider using one or more components of system 100 or system 600. Method 1000 includes attaching a component to a tissue site via a light switchable adhesive to form a bond between the component and the tissue site, at 1010. For example, the component may include or correspond to a compound film 150 or a medical device, such as a component (e.g., drape 632) of system 600. The tissue site may include or correspond to tissue 422 or tissue site 620, and the light switchable adhesive may include or correspond to light switchable adhesive 110. To illustrate, a patient or care provider applies a component, such as drape 632 or compound film 152, to tissue 422 or tissue site 620 with LSA 110 or LSA 496 while the LSA is in a first phase 142.

Method 1000 also includes applying first light to the light switchable adhesive to increase a bond strength of the bond between the component and the tissue site, at 1012. For example, the first light may include or correspond to first light 132. To illustrate, a light device 112 or a light source 114 provides first light 132 or third light 136 to the LSA 110 to change from the first phase 142 to the second phase 144, as illustrated in FIG. 1A, to increase bond strength, as illustrated in FIG. 3.

Method 1000 includes applying second light to the light switchable adhesive to decrease the bond strength of the bond between the component and the tissue site, at 1014. For example, the second light may include or correspond to second light 134 or third light 136. To illustrate, a light device 112 or a light source 114 provides light different from first light 132 to the LSA 110 to change from the second phase 144 to the third phase 146, as illustrated in FIG. 1A, to decrease bond strength, as illustrated in FIG. 3.

Method 1000 further includes removing the component from the tissue site, at 1016. For example, a patient care provider or patient removes the compound film or medical device, such as a bandage, wound closure device, wound dressing, etc., from the tissue 422 or tissue site 620. In a particular implementation, the medical device is removed after a period of time or after an indication is produced by the LSA, such as a color change, as described with reference to FIG. 1.

Thus, method 1000 describes a method of using a light switchable adhesive to attach a component to a tissue site. The light switchable adhesive enables medical devices to be repositionable and be more resistant to inadvertent contact as compared to conventional light switchable adhesives, thereby increasing usability and reducing waste and patient discomfort. Accordingly, the light switchable adhesives described herein may enable improved wound care and therapy, thereby advancing patient comfort and confidence in the treatment.

FIG. 11 illustrates a method 1100 of using a light switchable adhesive. The method 1100 may be performed at or by LSA 110, system 100 (e.g., LSA 110 thereof), system 600 (e.g., LSA 110 thereof), LSA 496, LSA 754, or LSA 810. Method 1100 includes receiving first light, at 1110. For example, the first light may include or correspond to first light 132 or third light 136. To illustrate, an LSA receives first light 132 or third light 136 from a light device 112 or a light source 114, as illustrated in FIG. 1A.

Method 1100 also includes transitioning from a first phase to a second phase, at 1112. For example, the first phase may include or correspond to first phase 142, and the second phase may include or correspond to second phase 144. To illustrate, the LSA transitions from the first phase 142 to the second phase 144 and increases in cross-linking and increases in peel strength as illustrated in FIGS. 2C and 3.

Method 1100 includes receiving second light different from the first light, at 1114. For example, the second light may include or correspond to second light 134 or third light 136. To illustrate, the LSA receives second light 134 or third light 136 from a light device 112 or a light source 114, as illustrated in FIG. 1A.

Method 1100 further includes transitioning from the second phase to a third phase, at 1116. For example, the third phase may include or correspond to the third state or phase 146, such as LSA 110 in FIG. 2D or LSA at t3 or t4 in FIG. 3. To illustrate, the LSA transitions from the second phase 144 to the third phase 146 and increases in cross-linking and decreases in peel strength as illustrated in FIGS. 2D and 3.

In some implementations, method 1100 further comprises transitioning from the third phase to a fourth phase. For example, the fourth state may include or correspond to the third state or phase 146 or a fourth state for phase, such as LSA 110 in FIG. 2D or LSA at t3 or t4 in FIG. 3. To illustrate, the LSA increases in cross-linking and decreases in peel strength further as illustrated in FIG. 3.

In some implementations, method 1100 further comprises providing a visual indication responsive to receiving light and/or transitioning. For example, the first photo initiator may indicate a color change from blue/green to translucent, white to purple, blue to purple, etc., after the first light has transitioned the LSA from the first phase to the second phase. As another example, the LSA may fluoresce blue when second light is being applied and may provide a yellow color shift (e.g., turn yellow in color) after the second light has transitioned the LSA from the second phase to the third phase.

Thus, method 1100 describes method of forming a compound film that includes light switchable adhesive and that is suitable for use with light switchable adhesive. The three or more phase light switchable adhesive enables medical devices to be repositionable and be more resistant to inadvertent contact as compared to conventional light switchable adhesives, thereby increasing usability and reducing waste and patient discomfort. Accordingly, the light switchable adhesives described herein may enable improved wound care and therapy, thereby advancing patient comfort and confidence in the treatment.

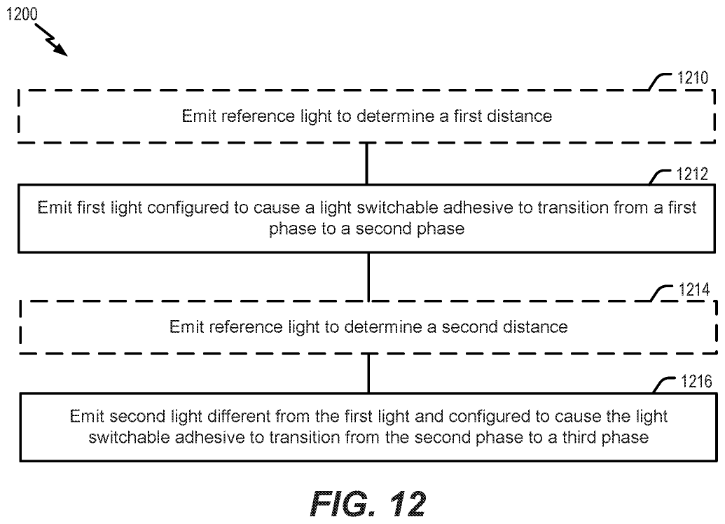

FIG. 12 illustrates a method 1200 of using a dual light device to activate a light switchable adhesive. The method 1200 may be performed at or by system 100 (e.g., light device 112 and/or light source 114 thereof), light source 618, light device 812, or light source 814.

Method 1200 optionally includes emitting reference light to determine a first distance, at 1210. For example, the reference light may include or correspond to light emitted by the reference light source 836. To illustrate, the reference light source 836 emits reference light which is analyzed by controller 842. The controller 842 determines a distance between the light device 812 and a surface (e.g., a surface of a component including LSA) based on the reference light and outputs a visual, audio, or haptic indication via indicator 844.

Method 1200 includes emitting first light configured to cause a light switchable adhesive to transition from a first phase to a second phase, at 1212. For example, the first light may include or correspond to first light 132 or third light 136, and the light switchable adhesive may include or correspond to LSA 110 or LSA 754. For example, the first phase may include or correspond to the first phase 142, and the second phase may include or correspond to the second phase 144. To illustrate, a light device 112 or a light source 114 provides first light 132 or third light 136 to LSA 110 to transition the LSA from the first phase 142 to the second phase 144, as illustrated in FIG. 1A.

Method 1200 optionally includes emitting reference light to determine a second distance, at 1214. For example, the reference light may include or correspond to light emitted by the reference light source 836. To illustrate, the reference light source 836 emits reference light which is analyzed by controller 842. The controller 842 determines a distance between the light device 812 and a surface (e.g., a surface of a component including LSA) based on the reference light and outputs a visual, audio, or haptic indication via indicator 844. The second distance may include or correspond to a distance that corresponds to second light which activates second photo initiators of the LSA. The second distance may be different from the first distance. As an illustrative example, the first distance, the second distance, or both, is about 20 mm. In other implementations, the distances are between 5 mm to 100 cm.

Method 1200 further includes emitting second light different from the first light and configured to cause the light switchable adhesive to transition from the second phase to a third phase, at 1216. For example, the second light may include or correspond to second light 134 or third light 136, and the third phase may include or correspond to third phase 146. To illustrate, a light device 112 or a light source 114 provides light different from first light 132 to LSA 110 to transition the LSA from the second phase 144 to the third phase 146, as illustrated in FIG. 1A. In some implementations, method 1200 further comprises coupling a cover film to the light switchable adhesive. For example, the cover film 198, 498 may by removably coupled to the LSA 110.

Thus, method 1200 describes method of activating a three or more phase light switchable adhesive. The three or more phase light switchable adhesive enables medical devices to be repositionable and be more resistant to inadvertent contact as compared to conventional light switchable adhesives, thereby increasing usability and reducing waste and patient discomfort. Accordingly, the light switchable adhesives described herein may enable improved wound care and therapy, thereby advancing patient comfort and confidence in the treatment.

It is noted that one or more operations described with reference to one of the methods of FIGS. 9-12 may be combined with one or more operations of another of FIGS. 9-12. For example, one or more operations of method 900 may be combined with one or more operations of method 1000. Additionally, or alternatively, one or more operations described above with reference to FIGS. 1A, 1B, 2A-2D, 3, 4A-4D, 6, and 7 may be combined with one or more operations of FIGS. 9-12, or a combination of FIGS. 9-12.

The above specification and examples provide a complete description of the structure and use of illustrative examples. Although certain aspects have been described above with a certain degree of particularity, or with reference to one or more individual examples, those skilled in the art could make numerous alterations to aspects of the present disclosure without departing from the scope of the present disclosure. As such, the various illustrative examples of the methods and systems are not intended to be limited to the particular forms disclosed. Rather, they include all modifications and alternatives falling within the scope of the claims, and implementations other than the ones shown may include some or all of the features of the depicted examples. For example, elements may be omitted or combined as a unitary structure, connections may be substituted, or both. Further, where appropriate, aspects of any of the examples described above may be combined with aspects of any of the other examples described to form further examples having comparable or different properties and/or functions, and addressing the same or different problems. Similarly, it will be understood that the benefits and advantages described above may relate to one example or may relate to several examples. Accordingly, no single implementation described herein should be construed as limiting and implementations of the disclosure may be suitably combined without departing from the teachings of the disclosure.

The previous description of the disclosed implementations is provided to enable a person skilled in the art to make or use the disclosed implementations. Various modifications to these implementations will be readily apparent to those skilled in the art, and the principles defined herein may be applied to other implementations without departing from the scope of the disclosure. Thus, the present disclosure is not intended to be limited to the implementations shown herein but is to be accorded the widest scope possible consistent with the principles and novel features as defined by the following claims. The claims are not intended to include, and should not be interpreted to include, means-plus- or step-plus-function limitations, unless such a limitation is explicitly recited in a given claim using the phrase(s) “means for” or “step for,” respectively.

1. A light switchable adhesive comprising:
one or more polymers;
first photo initiators configured to cause the one or more polymers to cross-link responsive to receiving first light; and
second photo initiators configured to cause the one or more polymers to cross-link responsive to receiving second light, the second photo initiators different from the first photo initiators.

2. The light switchable adhesive of claim 1, wherein the light switchable adhesive has at least three phases, each phase corresponding to a particular peel strength, and wherein the light switchable adhesive is configured to transition between a first two phases of the three phases based on activation of the first photo initiators and to transition between a second two phases of the three phases based on activation of the second photo initiators.

3. The light switchable adhesive of claim 2, wherein the light switchable adhesive has a second peel strength in the second phase that is greater than a first peel strength of the light switchable adhesive in the first phase, and wherein a third peel strength of the light switchable adhesive in the third phase is less than the second peel strength.

4. The light switchable adhesive of claim 1, wherein the one or more polymers include an Acrylate polymer, and wherein the light switchable adhesive comprises a polymer composition that includes the one or more polymers, the first photo initiators, and the second photo initiators.

5. The light switchable adhesive of claim 1, wherein the one or more polymers include urethane acrylate, methyl acrylate, silicone acrylate, polyether, polyurethane, or a combination thereof.

6. The light switchable adhesive of claim 1, wherein the first photo initiators have a peak absorbance between 750 nanometers (nm) to 860 nm, and wherein the second photo initiators have a peak absorbance between 200 nanometers (nm) to 400 nm. Canceled.

8. The light switchable adhesive of claim 1, wherein the first photo initiators comprise H-Nu-IR 780, H-Nu-IR 815, or both, and wherein the second photo initiators comprise Irgacure 819.

9. (canceled)

10. The light switchable adhesive of claim 1, wherein the light switchable adhesive has a peel strength of less than 7 Newtons per 25 millimeters (7 N/25 mm) on stainless steel at 180 degrees in a first phase, has a peel strength of greater 8 Newtons per 25 millimeters (8 N/25 mm) on stainless steel at an angle of 180 degrees in a second phase, has a peel strength of greater 8 Newtons per 25 millimeters (8 N/25 mm) on stainless steel at an angle of 180 degrees in a second phase, or a combination thereof.

11-12. (canceled)

13. The light switchable adhesive of claim 1, wherein the light switchable adhesive has an areal weight of 100 to 250 grams per square meter (gsm), has a moisture vapor transfer rate (MVTR) of greater than 250 grams per meter squared (g/m²), or both.

14. (canceled)

15. The light switchable adhesive of claim 1, wherein the light switchable adhesive has 7 to 11 millimeters of cone penetration as measured by an ISO 2173 cone penetration test with a 62.5 gram cone and a duration of 5 seconds.

16. The light switchable adhesive of claim 2, wherein:
the light switchable adhesive has a second level of cross-linking in the second phase that is greater than a first level of cross-linking in the first phase; and
the light switchable adhesive has a third level of cross-linking in the third phase that is greater than the second level of cross-linking.

17. A compound film comprising:
a first layer of a first polymer composition;
a second layer of a second polymer composition, the second layer removeably coupled to the first layer; and
a light switchable adhesive coupled to the second polymer layer, the light switchable adhesive comprising:
one or more polymers;
first photo initiators configured to cause the one or more polymers to cross-link responsive to receiving first light; and
second photo initiators configured to cause the one or more polymers to cross-link responsive to receiving second light, the second photo initiators different from the first photo initiators.

18. The compound film of claim 17, wherein:
the first layer is in direct contact with the second layer; and
the light switchable adhesive is in direct contact with the second layer.

19. Canceled.

20. The compound film of claim 17, wherein:
the first layer is configured to block or filter ultraviolet light, visible light, or both; and
the second layer is configured to pass ultraviolet light, visible light, or both.

21. (canceled)

22. The compound film of claim 17, wherein:
the second layer is configured to pass visible light, infrared light, or both; and
the first layer is configured to block or filter visible light.

23. (canceled)

24. The compound film of claim 17, wherein the first layer is included in a drape, a bandage, a wound closure device, a therapy system adhesive, or a combination thereof.

25-46. (canceled)

47. A method of using light switchable adhesive, the method comprising:
receiving, at a light switchable adhesive of a component, first light;
responsive to receiving the first light, transitioning, by the light switchable adhesive, from a first phase to a second phase;
receiving, at the light switchable adhesive, second light, the second light different from the first light; and
responsive to receiving the second light, transitioning, by the light switchable adhesive, from the second phase to a third phase.

48. The method of claim 47, further comprising:
after receiving the first light, increasing, by the light switchable adhesive, a bond strength between the component and a tissue site; and
after receiving the second light, decreasing, by the light switchable adhesive, a bond strength between the component and the tissue site.

49. The method of claim 47, further comprising:
prior to receiving the first light, bonding, by the light switchable adhesive, the component to a tissue site; and
after receiving the second light, debonding, by the light switchable adhesive, the component from the tissue site, wherein the third phase has a lower peel strength than the second phase.

50. The method of claim 47, further comprising, responsive to receiving the first light or the second light, changing color by the light switchable adhesive.

51-65. (canceled)
